(19) United States
(12) Patent Application Publication
(10) Pub. No.: US 2009/0318846 A1
(43) Pub. Date: Dec. 24, 2009

(54) METHODS AND APPARATUS FOR SURFACE ABLATION

(75) Inventors: Mark R. Prausnitz, Atlanta, GA (US); Mark G. Allen, Atlanta, GA (US); Jung-Hwan Park, Gyunggi-do (KR); Jeong Woo Lee, Atlanta, GA (US); Priya D. Gadiraju, Atlanta, GA (US)

Correspondence Address: TROUTMAN SANDERS LLP;BANK OF AMERICA PLAZA, 600 PEACHTREE STREET, N.E., SUITE 5200, ATLANTA, GA, 30308-2216, US

(73) Assignee: Georgia Tech Research Corporation, Atlanta, GA (US)

(21) Appl. No.: 12/130,728

(22) Filed: May 30, 2008

Related U.S. Application Data
(63) Continuation-in-part of application No. 11/597,969, filed on Aug. 15, 2007.
(60) Provisional application No. 60/940,719, filed on May 30, 2007.
(60) Provisional application No. 60/575,717, filed on May 28, 2004.

(51) Int. Cl. A61N 1/30; A61B 18/18

(52) U.S. Cl. 604/20; 606/41

(57) ABSTRACT

The various embodiments of the present invention relate generally to methods and apparatus for surface ablation. More particularly, various embodiments of the present invention are related to methods and apparatus for ablation of barrier surfaces, such as skin, to increase the permeability of the barrier surface. Embodiments of the present invention comprise rapid thermo-mechanical ablation of the skin by a microfluidic jet generated by an arc discharge to produce micron-scale holes localized to the stratum corneum, which increases skin permeability.

### RELATED APPLICATIONS

This application claims, under 35 U.S.C. § 119(e), the benefit of U.S. Provisional Application Ser. No. 60/940,719, filed 30 May 2007, and is a continuation-in-part of U.S. patent application Ser. No. 11/597,969 filed 15 Aug. 2007, which is a 35 U.S.C. § 371 U.S. National Stage Application of International Application Number PCT/US2005/019035 filed 31 May 2005, which claims, under 35 U.S.C. § 119(e), the benefit of U.S. Provisional Application Ser. No. 60/575,717, filed 28 May 2004, the entire contents and substance of which are hereby incorporated by reference as if fully set forth below.

### GOVERNMENT LICENSE RIGHTS

This invention was made with U.S. Government support under Grant No. DAAD19-00-1-0518 awarded by the U.S. Army. The U.S. Government has certain rights in the invention.

### TECHNICAL FIELD

The various embodiments of the present invention relate generally to methods and apparatus for surface ablation. More particularly, various embodiments of the present invention are related to methods and apparatus for ablation of barrier surfaces, such as skin, to increase the permeability of the barrier surface.

### BACKGROUND OF THE INVENTION

Transdermal drug delivery is an attractive method to administer drugs. Drug delivery across the skin circumvents enzymatic degradation of the drug in the gastrointestinal tract, poor intestinal absorption of the drug, the first-pass liver effect associated with oral drug delivery, and avoids the pain and inconvenience of injections. Furthermore, conventional oral or parenteral drug delivery are often not suitable for many protein, peptide, DNA, nucleic acid, small molecule, or other biotechnologically-based therapies currently proposed and envisioned. In addition, drug delivery across the skin readily permits sustained or modulated delivery from a passive or active patch, offering the capability to continuously control the delivery rate of the drug, in contrast to conventional methods that deliver a large, discrete bolus. For at least these reasons, transdermal drug delivery represents a multi-billion dollar market, which has a significant impact on medical and pharmaceutical industries.

Despite these advantages, transdermal drug delivery is difficult to achieve because of skin's highly impermeable outer layer, the stratum corneum. The stratum corneum is 10-20 μm thick and contains dead keratinocytes rich in the tough fibrous protein keratin that are held together by an intercellular matrix of neutral lipids. There are no blood vessels or nerves in stratum corneum. Below the stratum corneum is the viable epidermis, which is 50-100 μm thick and also contains no blood vessels, but has some nerves. Deeper still is the dermis, which measures 1-2 mm thick and contains a plexus blood vessels, lymphatics, and nerves. Thus, if an active agent can successfully traverse the stratum corneum barrier, the active agent generally can diffuse through the viable epidermis to the capillaries in the superficial dermis for absorption and systemic distribution. Local delivery to the skin may also be desirable to treat dermatological indications, to target vaccines or immunotherapeutics to immune cells in the skin, for cosmetic purposes, and other applications. For these reasons, most approaches to increase transdermal drug delivery have emphasized disruption of stratum corneum microstructure using chemical or physical methods.

Currently, transdermal drug delivery is limited to a small group of drugs that share a narrow set of common characteristics: low molecular weight (<500 Da), an octanol-water partition coefficient much greater than one, effective at low doses, and cause little or no skin irritation. Thus, few drugs can cross skin at useful therapeutic rates because the stratum corneum is an excellent barrier.

In order to overcome the stratum corneum barrier, a variety of chemical, physical, and mechanical techniques have been developed to create nanometer-scale disruptions to the structural organization of the stratum corneum, thereby increasing skin permeability. Chemical approaches, involving solvents, surfactants, and other compounds, have had varied success, where increased skin permeability has often been associated with increased skin irritation. Such chemical approaches have often been applicable only to small molecules and not macromolecules, such as peptides and proteins. Physical approaches, such as iontophoresis, electroporation, and ultrasound, have perturbed the stratum corneum structure and are more effective than chemical approaches in increasing skin permeability to a wider variety of macromolecules; however, the obtained increase in transdermal transport is still not therapeutically sufficient for many drugs under clinically acceptable conditions. This suggests that the approach to disrupting skin on the nanometer scale may be too mild.

In an effort to facilitate transdermal drug delivery of a broad range of compounds at therapeutically effective amounts, micron-scale skin disruption would make skin much more permeable, yet still be safe and well-tolerated by patients. Considering that almost all conventional drugs, proteins, nucleic acids (e.g., DNA), and vaccines are sub-micron in size, the creation of micron-sized holes in the stratum corneum would permit delivery of a broad range of compounds. Yet, micron-scale disruption is unlikely to have significant safety or cosmetic concerns. Consequently, a number of methods to disrupt stratum corneum on the micron scale have been developed, including thermal ablation, jet injection, and microneedles.

Thermal ablation of the skin involves disruption of the stratum corneum microstructure by rapidly heating the skin surface to thermally ablate micron-sized regions of stratum corneum. If the thermal pulse is short enough, there is a steep thermal gradient across the stratum corneum, so that deeper viable tissues are not heated. In this way, ablation is targeted to the stratum corneum so that living cells and nerves found deeper in the skin are not affected. Previous approaches to thermal ablation of the stratum corneum have involved heating filaments or an array of electrodes to generate Joule heating by passing a short, high-current electric pulse. Thermal ablation techniques have required long heating times of many milliseconds and can cause lasting damage to the skin with cosmetic effects that remain visible for many days.

Mechanical disruption of the skin has also been studied using a number of different techniques. Jet injection has existed for many decades and is based on high velocity penetration of a drug-containing liquid into the skin. Jet injection, however, is notoriously unreliable in the hands of patients, induces pain, and causes deep tissue damage in the form of bruising. Microneedles represent a newer technology that has recently received attention as a means to mechanically create conduits across the stratum corneum for minimally invasive delivery; however, penetration of microneedles is both invasive and cannot be localized to the stratum corneum, penetrating much deeper into the skin.

Accordingly, there is a need for methods and apparatus for surface ablation that can increase the permeability of barriers, such as skin. Further, there is a need for methods and apparatus that provide for transdermal transfer of a greater variety of active agents. Additionally, there is also a need for methods and apparatus that can aid in detecting and measuring analytes that are protected by a surface or barrier, particularly skin or other membranes. It is to the provision of such methods and apparatus that the various embodiments of the present invention are directed.

### SUMMARY OF THE INVENTION

Various embodiments of the present invention are directed generally to methods and apparatus for surface ablation. More particularly, various embodiments of the present invention are related to methods and apparatus for ablation of barrier surfaces, such as skin, to increase the permeability of the barrier surface.

Broadly described, an aspect of the present invention comprises an ablation system for a surface, comprising: an electric current generating system; a propulsion system in operative communication with the electric current generating system; and a medium, wherein the medium is propelled towards a surface by the propulsion system in response to an electric current generated by the electric current generating system, wherein the electric current does not contact the surface. The electric current generating system comprises at least one chamber containing the medium, the at least one chamber comprising at least two electrodes configured to generate the electrical current therebetween. In an embodiment of the present invention, the at least two electrodes of the at least one chamber are configured to permit an arc discharge therebetween. The propulsion system comprises the at least one chamber having a nozzle, wherein the medium is propelled from the at least one chamber through the nozzle upon generation of a current. In an embodiment of the present invention, the ablation system is capable of ablating the surface in less than about 100 microseconds. In an embodiment of the present invention, the ablation system further comprises an interface layer, wherein the interface layer is provided between the ablation system and the surface. In an embodiment of the present invention, the interface layer can comprise regions having different heat transfer properties. In an embodiment of the present invention, the surface is a biological surface. The ablation system of the present invention can further comprise at least one active agent, wherein the at least one active agent is delivered to the surface. In an embodiment of the present invention, the volume of each of the at least one chambers is less than about one milliliter. In an embodiment of the present invention, the area of the surface ablated by each of the at least one chambers comprises less than about one millimeter.

An aspect of the present invention comprises a method for ablating a surface, comprising: providing an ablation apparatus to a surface; generating an electric current with the ablation apparatus, wherein the electric current does not contact the surface; ejecting a medium from the ablation apparatus towards the surface; and ablating the surface. In an embodiment of the present invention, generating a current can comprise inducing an arc discharge. In an embodiment of the present invention, the surface can comprise a biological surface, wherein the biological surface is skin or a mucosal tissue. In an embodiment of the present invention, ablating the surface can comprise altering the stratum corneum. The method for ablating a surface can further comprise providing an interface layer between the ablation system and the surface, which can further comprise differentially transferring heat to different regions of the surface. The method for ablating a surface can further comprise delivering an active agent to a surface. In an embodiment of the present invention, ablating the surface can occur in less than about 100 microseconds.

An aspect of the present invention comprises an ablation system for a surface comprising an electric current generating system, a propulsion system, and a medium, wherein the electric current generated by the ablation system does not contact the surface. The electric current generating system comprises a chamber containing a medium, the chamber comprising at least two electrodes configured to permit an electrical current therebetween. The propulsion system comprises the chamber having a nozzle, wherein the medium is propelled from the chamber through the nozzle upon generation of a current.

An aspect of the present invention comprises an ablation system comprising an arc generating system, a propulsion system, and a medium. The arc generating system comprises a chamber containing a medium, the chamber comprising at least two electrodes configured to permit an arc to discharge therebetween. The propulsion system comprises the chamber having a nozzle, wherein the medium is propelled from the chamber through the nozzle upon discharge of an arc.

An embodiment of the present invention comprises an ablation system comprising: an ablation apparatus comprising at least one chamber having a nozzle, the at least one chamber comprising a first electrode and a second electrode, wherein the first electrode and second electrode are configured to permit generation of a current therebetween; and a medium contained within the at least one chamber. In an embodiment of the present invention, the ablation apparatus comprises one chamber. In another embodiment of the present invention, the ablation apparatus comprises a plurality of chambers. In the ablation system of the present invention, the generation of a current between the first electrode and second electrode can comprises an arc discharge. The medium can comprises many media, including but not limited to, a fluid, liquid, solid, solution, suspension, emulsion, gas, vapor, gel, dispersion, a flowable material, a multiphase material, or combination thereof. In an exemplary embodiment of the present invention, the medium comprises air, water, ethanol, saline, or combinations thereof. The at least one chamber of the ablation apparatus can comprises many shapes including but not limited to a post, a disk, a cone, a loop, or other geometrical shape. The at least one chamber of the ablation apparatus can have a volume of about 0.1 μl to about 10 μl.

The ablation system can be made by many methods know in the art, including but not limited to lamination techniques. The electrodes of the ablation system can be made of many materials, including but not limited to brass, nickel, platinum, titanium, tungsten, or other electrically conductive material having a high melting point. The electrodes of the ablation apparatus can be oriented on different sides of the at least one chamber. In an embodiment of the present invention, the electrodes are separated by a distance of 250 μm and are subjected to a voltage of about 100 V to about 150 V.

In an embodiment of the present invention, the ablation system is capable of ablating a surface in less than 100 μs. In an embodiment of the present invention, the ablation system is capable of ablating a surface in about 10 μs. In such an embodiment, the target surface for ablation can comprise many biological surfaces, including but not limited to skin or a mucosal tissue. The ablation system is capable of ablating a surface by a thermal and mechanical process.

An aspect of the present invention comprises an ablation system comprising: an ablation apparatus comprising at least one chamber having a nozzle, the at least one chamber comprising a first electrode and a second electrode, wherein the first electrode and second electrode are configured to permit generation of a current therebetween; a medium contained within the at least one chamber; and an interface layer. In an embodiment of the present invention, the ablation apparatus comprises a plurality of chambers. The generation of a current between the first electrode and second electrode can comprises an arc discharge. The medium of the present invention can comprise many media including but not limited to a fluid, liquid, solid, emulsion, solution, suspension, gas, vapor, gel, dispersion, a flowable material, a multiphase material, or combination thereof. The at least one chamber has a volume of about 0.1 μl to about 10 μl. The electrodes of the ablation apparatus can be made of many materials including but not limited to brass, nickel, platinum, titanium, tungsten, or other electrically conductive material having a high melting point. In an embodiment of the present invention, the electrodes are separated by a distance of 250 μm and are subjected to a voltage of about 100 V to about 150 V. The ablation system is capable of ablating a surface in less than 100 μs, and the ablation system is capable of ablating a surface in about 10 μs. The ablation system of the present invention can be used to ablate many surfaces, including but not limited to skin or a mucosal tissue.

In an embodiment of the present invention, an interface layer can be provided between the ablation system and a target surface. The interface layer comprises a layer of thermally conductive material having mechanical integrity. The interface layer comprises a layer of material at least partially lacking thermal conductivity but having mechanical integrity. The heat transfer properties of the interface layer control the amount of heat that is transferred across the interface layer from the medium ejected from the ablation device to the barrier. Heat transfer across the interface layer can be controlled by numerous of parameters, including but not limited to thermal conductivity of the interface layer and thickness of the interface layer. A hole in the interface layer could provide extensive heat transfer, because the medium is permitted to contact the barrier directly. In an embodiment of the present invention, the interface layer can comprise a plurality of holes, wherein the plurality of holes have a diameter of about 10 μm to about 100 μm. In another embodiment of the present invention, the interface layer can comprise a mosaic of thermally conductive regions and thermally insulative regions, wherein the regions of the mosaic possess mechanical integrity.

An aspect of the present invention comprises an active agent delivery system comprising: an ablation apparatus comprising at least one chamber having a nozzle, the at least one chamber comprising a first electrode and a second electrode, wherein the first electrode and second electrode are configured to permit generation of a current therebetween; a medium contained within the at least one chamber; and an active agent. In an embodiment of the present invention, the ablation apparatus comprises a plurality of chambers. The generation of a current between the first electrode and second electrode of the ablation apparatus can comprise an arc discharge. The active agent delivery system can comprise many media including but not limited to a fluid, liquid, solid, emulsion, solution, suspension, gas, vapor, gel, dispersion, a flowable material, a multiphase material, or combination thereof. The electrodes of the active agent delivery system can be made of brass, nickel, platinum, titanium, tungsten, or other electrically conductive material having a high melting point. In an embodiment of the active agent delivery system, the electrodes are separated by a distance of 250 μm and are subjected to a voltage of about 100 V to about 150 V. The ablation apparatus of the active agent delivery system is capable of ablating a surface in less than about 100 μs. The ablation apparatus of the active agent delivery system is capable of ablating a surface in less than about 10 μs. The active agent delivery system can ablate a surface comprising skin or a mucosal surface. In an embodiment of the active agent delivery system, the active agent is associated with the medium. The active agent can comprise an agent for gene therapy; nucleic acids; DNA; RNA; polynucleotides; peptides; proteins; amino acids; carbohydrates; viruses; antigens; immunogens; antibodies; chemical or biological materials or compounds that induce a desired biological or pharmacological effect; anti-infectives; antibiotics; antiviral agents; analgesics; analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations; potassium channel blockers; calcium channel blockers; beta-blockers; alpha-blockers; antiarrhythmics; antihypertensives; diuretics; antidiuretics; vasodilators comprising general coronary, peripheral and cerebral; central nervous system stimulants; vasoconstrictors; cough and cold preparations; decongestants; hormones; estradiol; steroids; progesterone and derivatives thereof; testosterone and derivatives thereof; corticosteroids; angiogenic agents; antiangeogenic agents; hypnotics; immunosuppressives; muscle relaxants; parasympatholytics; nicotine; psychostimulants; sedatives; tranquilizers, ionized and nonionized active agents; cells; compounds of either high or low molecular weight; and combinations thereof.

An aspect of the present invention comprises an active agent delivery system comprising: an ablation apparatus comprising at least one chamber having a nozzle, the at least one chamber comprising a first electrode and a second electrode, wherein the first electrode and second electrode are configured to permit generation of a current therebetween; a medium contained within the at least one chamber; an active agent; and an interface layer. In an embodiment of the present invention, the ablation apparatus comprises a plurality of chambers. The generation of a current between the first electrode and second electrode of the ablation apparatus can comprise an arc discharge. The active agent delivery system can comprise many media including but not limited to a fluid, liquid, solid, emulsion, solution, suspension, gas, vapor, gel, dispersion, a flowable material, a multiphase material, or combination thereof. The electrodes of the active agent delivery system can be made of brass, nickel, platinum, titanium, tungsten, or other electrically conductive material having a high melting point. In an embodiment of the active agent delivery system, the electrodes are separated by a distance of 250 μm and are subjected to a voltage of about 100 V to about 150 V. The ablation apparatus of the active agent delivery system is capable of ablating a surface in less than about 100 μs. The ablation apparatus of the active agent delivery system is capable of ablating a surface in less than about 10 μs. The active agent delivery system can ablate a surface comprising skin or a mucosal surface. In an embodiment of the active agent delivery system, the active agent is associated with the medium. The active agent can comprise an agent for gene therapy; nucleic acids; DNA; RNA; polynucleotides; peptides; proteins; amino acids; carbohydrates; viruses; antigens; immunogens; antibodies; chemical or biological materials or compounds that induce a desired biological or pharmacological effect; anti-infectives; antibiotics; antiviral agents; analgesics; analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations; potassium channel blockers; calcium channel blockers; beta-blockers; alpha-blockers; antiarrhythmics; antihypertensives; diuretics; antidiuretics; vasodilators comprising general coronary, peripheral and cerebral; central nervous system stimulants; vasoconstrictors; cough and cold preparations; decongestants; hormones; estradiol; steroids; progesterone and derivatives thereof; testosterone and derivatives thereof; corticosteroids; angiogenic agents; antiangeogenic agents; hypnotics; immunosuppressives; muscle relaxants; parasympatholytics; nicotine; psychostimulants; sedatives; tranquilizers, ionized and nonionized active agents; cells; compounds of either high or low molecular weight; and combinations thereof.

In an embodiment of the active agent delivery system, the interface layer is provided between the ablation system and a target surface. The interface layer comprises a layer of thermally conductive material having mechanical integrity. The interface layer comprises a layer of material at least partially lacking thermal conductivity but having mechanical integrity. The interface layer can also comprise a plurality of holes, wherein the plurality of holes have a diameter of about 10 to about 100 μm. In an embodiment of the active agent delivery system, the interface layer comprises a mosaic of thermally conductive regions and thermally insulative regions, wherein the regions of the mosaic possess mechanical integrity.

An aspect of the present invention comprise an active agent delivery system comprising: an ablation apparatus comprising at least one chamber having a nozzle, the at least one chamber comprising a first electrode and a second electrode, wherein the first electrode and second electrode are configured to permit generation of a current therebetween; a medium contained within the at least one chamber; and a formulation comprising at least one active agent. In an embodiment of the present invention, the ablation apparatus comprises a plurality of chambers. The generation of a current between the first electrode and second electrode of the ablation apparatus can comprise an arc discharge. The active agent delivery system can comprise many media including but not limited to a fluid, liquid, solid, emulsion, solution, suspension, gas, vapor, gel, dispersion, a flowable material, a multiphase material, or combination thereof. The electrodes of the active agent delivery system can be made of brass, nickel, platinum, titanium, tungsten, or other electrically conductive material having a high melting point. In an embodiment of the active agent delivery system, the electrodes are separated by a distance of 250 μm and are subjected to a voltage of about 100 V to about 150 V. The ablation apparatus of the active agent delivery system is capable of ablating a surface in less than about 100 μs. The ablation apparatus of the active agent delivery system is capable of ablating a surface in less than about 10 μs. The active agent delivery system can ablate a surface comprising skin or a mucosal surface. In an embodiment of the active agent delivery system, the active agent is associated with the formulation.

In an embodiment of the present invention, the formulation comprising at least one active agent comprises a patch. The active agent can comprises an agent for gene therapy; nucleic acids; DNA; RNA; polynucleotides; peptides; proteins; amino acids; carbohydrates; viruses; antigens; immunogens; antibodies; chemical or biological materials or compounds that induce a desired biological or pharmacological effect; anti-infectives; antibiotics; antiviral agents; analgesics; analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations; potassium channel blockers; calcium channel blockers; beta-blockers; alpha-blockers; antiarrhythmics; antihypertensives; diuretics; antidiuretics; vasodilators comprising general coronary, peripheral and cerebral; central nervous system stimulants; vasoconstrictors; cough and cold preparations; decongestants; hormones; estradiol; steroids; progesterone and derivatives thereof; testosterone and derivatives thereof; corticosteroids; angiogenic agents; antiangeogenic agents; hypnotics; immunosuppressives; muscle relaxants; parasympatholytics; nicotine; psychostimulants; sedatives; tranquilizers, ionized and nonionized active agents; cells; compounds of either high or low molecular weight; and combinations thereof.

An aspect of the present invention comprises an active agent delivery system comprising: an ablation apparatus comprising at least one chamber having a nozzle, the at least one chamber comprising a first electrode and a second electrode, wherein the first electrode and second electrode are configured to permit generation of a current therebetween; a medium contained within the at least one chamber; a formulation comprising at least one active agent; and an interface layer. In an embodiment of the present invention, the ablation apparatus comprises a plurality of chambers. The generation of a current between the first electrode and second electrode of the ablation apparatus can comprise an arc discharge. The active agent delivery system can comprise many media including but not limited to a fluid, liquid, solid, emulsion, solution, suspension, gas, vapor, gel, dispersion, a flowable material, a multiphase material, or combination thereof. The electrodes of the active agent delivery system can be made of brass, nickel, platinum, titanium, tungsten, or other electrically conductive material having a high melting point. In an embodiment of the active agent delivery system, the electrodes are separated by a distance of 250 μm and are subjected to a voltage of about 100 V to about 150 V. The ablation apparatus of the active agent delivery system is capable of ablating a surface in less than about 100 μs. The ablation apparatus of the active agent delivery system is capable of ablating a surface in less than about 10 μs. The active agent delivery system can ablate a surface comprising skin or a mucosal surface.

In an embodiment of the active agent delivery system, the active agent is associated with the formulation. In an embodiment of the present invention, the formulation comprising at least one active agent comprises a patch. The active agent can comprises an agent for gene therapy; nucleic acids; DNA; RNA; polynucleotides; peptides; proteins; amino acids; carbohydrates; viruses; antigens; immunogens; antibodies; chemical or biological materials or compounds that induce a desired biological or pharmacological effect; anti-infectives; antibiotics; antiviral agents; analgesics; analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations; potassium channel blockers; calcium channel blockers; beta-blockers; alpha-blockers; antiarrhythmics; antihypertensives; diuretics; antidiuretics; vasodilators comprising general coronary, peripheral and cerebral; central nervous system stimulants; vasoconstrictors; cough and cold preparations; decongestants; hormones; estradiol; steroids; progesterone and derivatives thereof; testosterone and derivatives thereof; corticosteroids; angiogenic agents; antiangeogenic agents; hypnotics; immunosuppressives; muscle relaxants; parasympatholytics; nicotine; psychostimulants; sedatives; tranquilizers, ionized and nonionized active agents; cells; compounds of either high or low molecular weight; and combinations thereof. In an embodiment of the present invention, the ablation apparatus can be associated with the formulation (e.g., a patch).

In an embodiment of the active agent delivery system comprising an interface layer, the interface layer is provided between the ablation apparatus and a target surface. The interface layer can comprise a layer of thermally conductive material having mechanical integrity. The interface layer can comprise a layer of material at least partially lacking thermal conductivity but having mechanical integrity. The interface layer comprises a plurality of holes, wherein the plurality of holes have a diameter of about 10 μm to about 100 μm. The interface layer can comprise a mosaic of thermally conductive regions and thermally insulative regions, wherein the regions of the mosaic possess mechanical integrity.

An aspect of the present invention comprises a method for ablating a surface comprising: a) providing to a surface an ablation apparatus comprising at least one chamber having a nozzle, the at least one chamber comprising a first electrode and a second electrode, wherein the first electrode and second electrode are configured to permit the discharge of an arc therebetween and a medium contained within the at least one chamber; b) generating a current between the first and second electrode; and c) ejecting the medium contained with the at least one chamber through the nozzle in the direction of the surface. In an embodiment of the present invention, the surface can be a biological surface. In an embodiment of the present invention, the biological surface is a tissue, wherein the tissue is a human tissue, an animal tissue, or a plant tissue, and wherein the tissue is skin, a dermal structure, a mucosal tissue, a membrane, or an organ. In an embodiment of the method for ablating a surface, providing to a surface an ablation apparatus can comprise contacting an ablation apparatus with a surface. In an embodiment of the method for ablating a surface, generating a current between the first and second electrode can comprise inducing an arc discharge between the first and second electrode.

In an embodiment of the present invention, the method for ablating a surface can further comprise d) transferring the energy of the medium to the surface, wherein the energy is thermal energy. In an embodiment of the present invention the method for ablating a surface can further comprise d) contacting the medium with a surface; and e) transferring the energy of the medium to the surface, wherein the energy is thermal energy and mechanical energy. In yet another embodiment of the present invention the method for ablating a surface can further comprise prior to step b) providing an interface layer to the surface.

An aspect of the present invention comprises a method for increasing the permeability of a barrier comprising: a) providing to a barrier an ablation apparatus comprising at least one chamber having a nozzle, the at least one chamber comprising a first electrode and a second electrode, wherein the first electrode and second electrode are configured to permit the discharge of an arc therebetween and a medium contained within the at least one chamber; b) generating a current between the first and second electrode; c) ejecting the medium contained with the at least one chamber through the nozzle in the direction of the barrier; and d) increasing the permeability of the barrier. In an embodiment of the present invention, the barrier is a biological barrier. In an embodiment of the present invention, the biological barrier is a tissue, wherein the tissue a human tissue, an animal tissue, or a plant tissue, and wherein the tissue is skin, a dermal structure, a mucosal tissue, a membrane, or an organ. In an embodiment of the method for increasing the permeability of a barrier, providing to a barrier an ablation apparatus can comprise contacting an ablation apparatus with a barrier. In an embodiment of the method for increasing the permeability of a barrier, generating a current between the first and second electrode can comprise inducing an arc discharge between the first and second electrode.

In an embodiment of the present invention, the method for increasing the permeability of a barrier can further comprise d) transferring the energy of the medium to the surface, wherein the energy is thermal energy. In an embodiment of the present invention, the method for increasing the permeability of a barrier can further comprise d) contacting the medium with a surface; and e) transferring the energy of the medium to the surface, wherein the energy is thermal energy and mechanical energy. In an embodiment of the present invention, the method for increasing the permeability of a barrier comprises increasing the permeability of the barrier by creating holes in the surface of the barrier, wherein the barrier is the stratum corneum. In yet another embodiment of the present invention, the method for increasing the permeability of a barrier can further comprise prior to step b) providing an interface layer to the surface

An aspect of the present invention comprises a method of delivery of an active agent comprising: a) providing to a surface an ablation apparatus comprising at least one chamber having a nozzle, the at least one chamber comprising a first electrode and a second electrode, wherein the first electrode and second electrode are configured to permit the discharge of an arc therebetween and a medium contained within the at least one chamber; b) generating a current between the first and second electrode; c) ejecting the medium contained with the at least one chamber through the nozzle in the direction of the surface; d) increasing the permeability of the surface; and e) delivering an active agent to the surface. In an embodiment of the present invention, the surface is a tissue, including but not limited to, skin, a dermal structure, a mucosal tissue, a membrane, or an organ. In an embodiment of the method of delivery of an active agent, the surface is the stratum corneum.

In an embodiment of the method of delivery of an active agent, providing to a surface an ablation apparatus can comprise contacting an ablation apparatus with a surface. In an embodiment of the method of delivery of an active agent, generating a current between the first and second electrode can comprise inducing an arc discharge between the first and second electrode. The method of delivery of an active agent can further comprise after step c), contacting the medium with a surface; and transferring the energy of the medium to the surface, wherein the energy is thermal energy and mechanical energy.

In an embodiment of the method of delivery of an active agent, increasing the permeability of a surface comprises creating holes in the surface. The active agent in the method of delivery of an active agent can comprise an agent for gene therapy; nucleic acids; DNA; RNA; polynucleotides; peptides; proteins; amino acids; carbohydrates; viruses; antigens; immunogens; antibodies; chemical or biological materials or compounds that induce a desired biological or pharmacological effect; anti-infectives; antibiotics; antiviral agents; analgesics; analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations; potassium channel blockers; calcium channel blockers; beta-blockers; alpha-blockers; antiarrhythmics; antihypertensives; diuretics; antidiuretics; vasodilators comprising general coronary, peripheral and cerebral; central nervous system stimulants; vasoconstrictors; cough and cold preparations; decongestants; hormones; estradiol; steroids; progesterone and derivatives thereof; testosterone and derivatives thereof; corticosteroids; angiogenic agents; antiangeogenic agents; hypnotics; immunosuppressives; muscle relaxants; parasympatholytics; nicotine; psychostimulants; sedatives; tranquilizers, ionized and nonionized active agents; cells; compounds of either high or low molecular weight; and combinations thereof. In an embodiment of the method of delivery of an active agent, delivering an active agent to the surface comprises delivering an active agent across the skin. The method of delivery of an active agent can further comprising prior to step b) providing an interface layer to the surface

An aspect of the present invention comprises a method of sampling an analyte contained by a barrier comprising: a) providing an ablation apparatus comprising at least one chamber having a nozzle, the at least one chamber comprising a first electrode and a second electrode, wherein the first electrode and second electrode are configured to generate a current therebetween and a medium contained within the at least one chamber; b) generating a current between the first and second electrode; c) ejecting the medium contained with the at least one chamber through the nozzle in the direction of the barrier; d) increasing the permeability of the barrier; and e) sampling at least one analyte contained by the barrier. In an embodiment of the method of sampling an analyte contained by a barrier, the barrier is a biological barrier, wherein the biological barrier is a tissue, wherein the tissue is a human tissue, an animal tissue, or a plant tissue, wherein the tissue is skin, a dermal structure, a mucosal tissue, a membrane, or an organ.

In an embodiment of the method of sampling an analyte contained by a barrier, providing to a barrier an ablation apparatus comprises contacting an ablation apparatus with a barrier. In an embodiment of the method of sampling an analyte contained by a barrier, generating a current between the first and second electrode comprises inducing an arc discharge between the first and second electrode. The method of sampling an analyte contained by a barrier can further comprising after step c), contacting the medium with a barrier; and transferring the energy of the medium to the barrier, wherein the energy is thermal energy and mechanical energy. In an embodiment of the method of sampling an analyte contained by a barrier, increasing the permeability of the barrier comprises creating holes in the surface of the barrier, wherein the barrier is the stratum corneum. The method of sampling an analyte contained by a barrier can further comprise prior to step b) providing an interface layer between the ablation apparatus and the barrier.

The at least one analyte of the method of sampling an analyte contained by a barrier of comprise molecules and substances of diagnostic interest, natural and therapeutically introduced metabolites, hormones, amino acids, peptides, proteins, polynucleotides; cells electrolytes, metal ions, suspected drugs of abuse, enzymes, tranquilizers, anesthetics, analgesics, anti-inflammatory agents, immunosuppressants, antimicrobials, muscle relaxants, sedatives, antipsychotic agents, antidepressants, antianxiety agents, small drug molecules, glucose, cholesterol, high density lipoproteins, low density lipoproteins, triglycerides, diglycerides, monoglycerides, bone alkaline phosphatase (BAP), prostate-Specific-Antigen (PSA), antigens, bilirubin, lactic acid, pyruvic acid, alcohols, fatty acids, glycols, thyroxine, estrogen, testosterone, progesterone, theobromine, galactose, urea, uric acid, alpha amylase, choline, L-lysine, sodium, potassium, copper, iron, magnesium, calcium, zinc, citrate, ammonia, lead, lithium, morphine, morphine sulfate, heroin, insulin, interferons, erythropoietin, fentanyl, cisapride, risperidone, infliximab, heparin, steroids, neomycin, nitrofurazone, betamethasone, clonidine, acetic acid, alkaloids, salicyclates, and acetaminophen. The method of sampling an analyte contained by a barrier can further comprise analyzing, measuring, or detecting the at least one analyte.

Other aspects and features of embodiments of the present invention will become apparent to those of ordinary skill in the art, upon reviewing the following description of specific, exemplary embodiments of the present invention in conjunction with the accompanying figures.

### BRIEF DESCRIPTION OF DRAWINGS

FIG. 1 illustrates a schematic of an ablation system.

FIG. 2 A illustrates an exploded view of an ablation system.

FIGS. 2 B-C illustrate a side view of an ablation system having a single chamber and an ablation system having an array of chambers, respectively.

FIGS. 3 A-B illustrate schematics of an ablation system having an interface layer.

FIG. 4 graphically depicts skin permeability to calcein after thermal treatment for various exposure times at different temperatures.

FIGS. 5 A-H provide confocal microscopy images of histological sections of stratum corneum stained with Nile Red after thermal exposure for 1 s at different temperatures.

FIG. 6 illustrates a schematic diagram of a wireless inductive heating system for micro-ablation of stratum corneum.

FIGS. 7 A-B provide an image of an array of micro-heating elements designed as hollow posts, and a cross-section of the region labeled A-A′.

FIG. 8 A provides an image of thermal paper exposed to a hollow-post micro-heater array.

FIG. 8 B graphically illustrates induction heating characteristics of the hollow-post array after excitation as a function of time at two frequencies.

FIGS. 9 A-B provide scanning electron micrographs of human cadaver skin ablated by hollow-post micro-heater inductive heating. FIG. 9 A is a top view, and FIG. 9 B is an angled view.

FIGS. 10 A-B are schematics of proximity mode inclined UV lithography. FIG. 10A shows a front-side exposure, and FIG. 10 B shows a reverse-side exposure through a transparent substrate and a gap layer.

FIGS. 11 A-B is an optical micrograph (A) and scanning electron micrograph (B) of sections of arrays of micro-nozzles prepared by proximity-mode inclined UV lithography.

FIGS. 12 A-C provide images of proximity mode inclined rotational UV lithography: (A) front-side exposure, (B) a reverse-side exposure with a 200 μm thick glass gap, and (C) reverse side exposure with a 200 μm thick glass gap and an additional vertical exposure for a central column.

FIGS. 13 A-C illustrate contact mode inclined rotational UV lithography: (A) front-side exposure, (B) a reverse-side exposure, and (C) reverse side exposure with multiple inclined angles.

FIG. 14 is a schematic of nozzles fabricated using proximity mode inclined rotational exposure with a continuously varying gap.

FIG. 15 provides an image of a fabricated micronozzle array with various orifice sizes resulting from a continuously varying gap.

FIG. 16 is a photomicrograph of a microheater array: (left) the whole array and (right) a magnified view of the probing pads on the left and the heaters on the right.

FIG. 17 provides an image of an integrated microablation system with micro-nozzles bonded on top of a micro-heater array.

FIG. 18 is a micro-ablation device with reservoirs filled with viscous ethanol gel.

FIG. 19 graphically depicts human cadaver skin permeability to calcein for intact skin (black bar), for skin contacted with a heated microdevice (dark gray bar) and for skin contacted with an ethanol-filled, heated microdevice (light gray bar).

FIGS. 20 A-B graphically depicts micro-reservoir temperature (A) and skin permeability (B) associated with the millisecond-long micro-ablation system.

FIG. 21 is a schematic of a cross-sectional view of a microdevice for arc-based ablation of the skin oriented with the nozzle directed out of the page.

FIG. 22 is a schematic of a cross-sectional view of a microdevice for arc-based ablation of the skin oriented with the ejectate nozzle directed upwards.

FIG. 23 provides an image of a microjet expelled from the nozzle of the arc-based microdevice.

FIG. 24 A is an en face image of porcine cadaver skin exposed to localized arc-based ablation.

FIGS. 24 B-D are histological cross-sectional images of porcine cadaver skin exposed to localized arc-based ablation at different levels of magnification.

FIGS. 25 A-B provide a flow chart of the method of force sensing for arc-based ablation (A) and a graphical image of the force measured (B).

FIG. 26 graphically illustrates the permeability of human cadaver skin to calcein after arc-based ablation.

FIG. 27 graphically depicts human cadaver skin permeability to calcein as a function of formulation filled into a microreservoir.

FIG. 28 graphically illustrates human cadaver skin permeability to calcein versus ablation microdevice reaction force, which is a measure of the microjet ejectate force.

FIGS. 29 A-B are images of the surface of pig cadaver skin before (A) and after (B) delivering sulforhodamine for 12 h.

FIGS. 30 A-C are histological images of untreated (top) and ablated (bottom) skin samples. Column A are brightfield microscopy images. Column B are fluorescent microscopy images. Column C are samples stained with hematoxylin and eosin.

### DETAILED DESCRIPTION

The various embodiments of the present invention relate generally to methods and apparatus for surface ablation. Particularly, the various embodiments of the present invention relate to methods and apparatus to increase the permeability of a barrier surface. More particularly, the various embodiments of the present invention relate to increasing the permeability of a biological surface for the delivery of an active agent.

An embodiment of the present invention comprises an ablation system comprising a current generating system, a propulsion system, and a medium. The current generating system of the ablation system can comprise a chamber containing a medium, the chamber comprising at least two electrodes configured to generate an electrical current therebetween. The propulsion system of the ablation system can comprise the chamber having a nozzle, wherein the medium is propelled from the chamber through the nozzle upon generation of an electrical current. An aspect of this embodiment comprises an electrically conductive medium.

An embodiment of the present invention comprises an ablation system comprising an arc generating system, a propulsion system, and a medium. The arc-generating system of the ablation system can comprise a chamber containing a medium, the chamber comprising at least two electrodes configured to permit an arc to discharge therebetween. The propulsion system of the ablation system can comprise the chamber having a nozzle, wherein the medium is propelled from the chamber through the nozzle upon discharge of an arc.

An aspect of the present invention comprises apparatus and methods for surface ablation. The methods and apparatus of the present invention can be used on many surfaces, including but not limited to biological and non-biological surfaces. Exemplary embodiments of biological surfaces include but are not limited to membranes and tissues of a human, an animal, a plant, and other living organisms, among others. In an exemplary embodiment, a tissue comprises skin, a dermal structure, a mucosal tissue, a membrane, and an organ, among others.

As used herein, “ablation” means the controlled removal of a region of the barrier, due to the actions of an activated ablation system in proximity with the barrier. Though not wishing to be bound by any particular theory, it is believed that the thermal and mechanical energy provided by the ablation system, optionally in combination with the composition of the medium or other ablation materials, cause the barrier or components of the barrier to be rapidly damaged at the target site.

Various embodiments of the present invention comprise apparatus and methods for surface ablation of a biological tissue, increasing the permeability of the biological tissue. In an embodiment of the present invention, apparatus and methods for surface ablation of a biological tissue comprise forming holes in a biological surface. In an embodiment of the present invention, apparatus and methods for surface ablation of a biological surface further comprise delivering an active agent to a biological surface by way of the holes created in the biological surface. In an exemplary embodiment of the present invention, the biological surface is the skin. An exemplary embodiment of the present invention comprises forming holes in the stratum corneum layer of the skin. Upon ablation of the stratum corneum, an active agent can be delivered to the tissue beneath the stratum corneum.

Referring now to the Figures, wherein like reference numerals represent like parts throughout the several views, exemplary embodiments of the present invention will be described in detail. Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented.

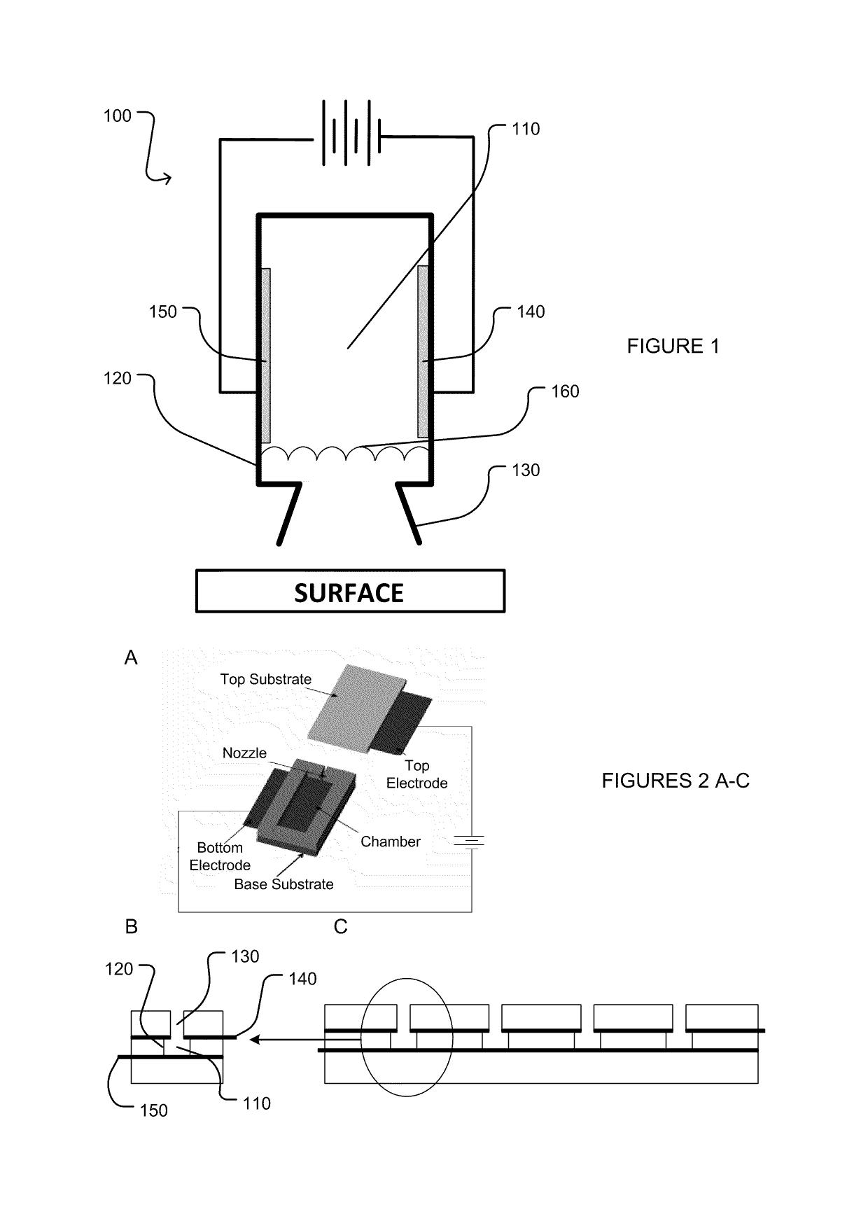

As illustrated in FIG. 1, an aspect of the present invention comprises an ablation system 100 comprising an ablation apparatus 110 comprising at least one chamber 120 having a nozzle 130, the at least one chamber 120 comprising a first electrode 140 and a second electrode 150, wherein the first electrode 140 and second electrode 150 are configured to permit the discharge of an arc therebetween; and a medium 160 contained with the at least one chamber 120.

According to various embodiments of the present invention, ablation of a surface can be accomplished by a thermal process, a mechanical process, or combinations thereof. Various embodiments of the present invention comprise apparatus and methods of using an ablation system also referred to herein as a microfluidic arc discharge ejector. An exemplary embodiment of the present invention comprises a microfluidic arc discharge ejector for the ablation of the skin. An arc discharge ejector propels a medium 160 contained within the at least one chamber 120 and located between the first electrode 140 and a second electrode 150 through a nozzle 130 at high velocity by passing high electric current between the first electrode 140 and the second electrode 150, creating arc discharge phenomena. This high velocity jet of medium can create holes in the target surface. In an embodiment of the present invention, a surface can be ablated by combined thermal and mechanical ablation methods operating over extremely short time scales. In an embodiment of the present invention, the holes are about 1 μm to about 1 cm in size. In an exemplary embodiment of the present invention, the holes are about 10 μm to about 1 mm in size or 10 μm to 100 μm in size.

The thermo-mechanical ablation apparatus and methods of the present invention comprise accelerating a microfluidic jet of a medium 160 from a chamber. As referred to herein, the medium 160 ejected from the at least one chamber may be referred to as an ejectate. In an embodiment of the present invention, an ablation system can comprise many media, including but not limited to, fluids, liquids, solids, emulsions, solutions, suspensions, gases, vapors, gels, dispersions, a flowable material, a multiphase material, or combination thereof. In an exemplary embodiment of the present invention, the medium can comprise air, water, deionized water, ethanol, saline, an ethanol-saline mixture, phosphate buffered saline (PBS), tris-buffered saline (TBS), an isotonic solution, an electrically conductive medium, hydrophobic liquids, hydrophilic liquids, methanol, organic compounds, alcohols, ketones, aldehydes, mixtures, or combinations thereof, among others. In an embodiment of the present invention, a media formulation can comprise a media approved by the Food and Drug Administration (FDA). In an embodiment of the present invention, the medium can further comprise impurities that increase the electrical conductivity of the medium, such as a dopant. Exemplary embodiments of dopants comprise carbon black particles, graphite, aluminum, salt, or combinations thereof, among others.

An ablation system 100 comprising an ablation apparatus 110 comprises at least one chamber 120. In an embodiment of the present invention, the ablation apparatus 110 comprises one chamber 120. In an embodiment of the present invention, the ablation apparatus 110 comprises a plurality of chambers 120. Each ablation system 100 is a separate element and thus, can be positioned in many locations. In an embodiment of the present invention, a plurality of chambers may have a variety of arrangements, for example, an array or a desired pattern. In an embodiment of the present invention, an array can comprise about two chambers to about one thousand (1,000) chambers. Edge-to-edge spacing of the chambers can range from about 0 to about 1 cm or about 0 to about 1 mm or about 10 μm to about 100 μm. The plurality of chambers may be arranged such that chambers having the same characteristics, such as electrode spacing or composition of the medium contained, are arranged together to provide an area of the ablation system that operates under one set of conditions, and another area of the ablation system comprises chambers having different characteristics so that in operation, one area under one set of conditions would discharge and another area would not. The plurality of chambers may be arranged so that chambers having one characteristic are alternated with chambers having a different characteristic, such as electrode spacing and composition of the medium contained. The plurality of chambers may be in contact with one another by a structure such as a plate attached to the base ends of the chambers, or by wires, or by a reservoir containing a surplus of medium.

The at least one chamber 120 of the ablation apparatus 110 can have many shapes and sizes. The at least one chamber 120 of the ablation apparatus 110 may be made in many desired shapes designed for a specific application. The at least one chamber 120 can be designed with different materials and geometries to produce different thermal and mechanical responses. For example, the shape of the at least one chambers may be a post, a disk, a cone, a loop, or other geometries. The volume of the at least one chamber 120 can be altered by varying the thickness of the chamber walls and/or the area of the at least one chamber 120. In an embodiment of the present invention, the at least one chamber 120 can have a volume of about 1 nl to about 1 ml or about 1 μl to about 100 μl or about 1 μl to about 10 μl. In an exemplary embodiment of the present invention, the at least one chamber 120 has a volume of 1 μl.

In an embodiment of the present invention, the ablation system 100 can further comprise a substrate upon which the ablation system 110 can be mounted. In an embodiment of the present invention, the substrate can comprise an insulating element. The insulating element acts as an insulator and prevents the transfer of heat from the heated portions of an ablation apparatus or from one or more ablation apparatus. The insulator can be made of many materials that provide thermal insulation, and is generally a non-conductor. Insulators of the present invention include, but are not limited to, Mylar, Kapton (polyimide), polyurethane, liquid crystal polymer, and epoxy, among others.

In an embodiment of the present invention, the ablation apparatus 110 comprising at least one chamber 120 may comprise a microfabricated device. There are several advantages in utilizing a microfabricated device to ablate a biological surface, including: (1) to minimize the skin hole size (submicron to microns to millimeters) with microfabricated devices; (2) to control the hole geometry; (3) to minimize infection through skin hole by reducing hole size, (4) to minimize pain by reducing hole size and the number of ablated spots, and by increasing the response of the microdevice; (5) to increase the integration density of holes by increasing the number of micro-spots in the unit area, (6) to make all-in-one device by integrating drug matrix with microdevices; (7) to increase skin contact with micro-units by fabricating microdevices on the top of a three-dimensional structure; (8) to encapsulate ablation system units in the transdermal patch or position ablation system units on the patch surface; and (9) to minimize pain by escaping direct contact with the ablation system. In an embodiment of the present invention, an ablation system 100 of the present invention may be utilized in combination with methods and devices for thermal treatment as described in U.S. patent application Ser. No. 11/597,969, International Application Number PCT/US2005/019035, and U.S. Provisional Patent Application No. 60/575,717, each of which are hereby incorporated by reference in its entirety.

In an embodiment of the present invention, an ablation apparatus 110 can be made by laser processing techniques, lamination techniques, lithography, molding, or machining techniques, among others. In an embodiment of the present invention, an ablation apparatus 110 can be fabricated using various micromachining techniques that enable patterning and etching of sub-micron geometries in a variety of materials. The ablation apparatus 110 of the present invention may be made of many materials, including but not limited to metal, non-metals, ceramics, polymers, organics, inorganics, composites, or combinations thereof. In an exemplary embodiment of the present invention, the ablation apparatus 110 can be made in a series of Mylar layers by laser processing, which are then laminated together by micro lamination techniques.

The ablation system 100 of the present invention propels a medium 160 from the at least one chamber 120 of the ablation apparatus 110 by an electrically-driven arc across the medium 160 contained within the at least one chamber 120 positioned between a first electrode 140 and a second electrode 150. The ablation system 100 utilizes the concept of generating heat rapidly within the ablation apparatus by passing a current through the medium. This current generation can be accomplished by generating an arc—applying a high voltage pulse across closely spaced electrodes. This discharge of high current through the medium 160 propels the medium 160 in the form of a jet through the nozzle 130 of the at least one chamber 120 at a high velocity. This high velocity jet is then used to create holes in the target surface.

The electrodes of the present invention may be made of many materials, including but not limited to brass, nickel, platinum, titanium, tungsten, copper, chromium, other electrically conductive materials optionally having a high melting point, or combinations thereof. A high melting point exceeds about 500° C. or about 1000° C. or about 1500° C. or about 2000° C. The electrodes of the present invention can be placed in many spatial orientations in the chamber. In an embodiment of the present invention, the first electrode 140 and the second electrode 150 can be placed on different sides of the at least one chamber 120 containing the medium 160. In an embodiment of the present invention, the first electrode 140 and the second electrode 150 can be placed on the same side of the at least one chamber 120 with a small gap between the electrodes. In an embodiment of the present invention, the first electrode 140 and the second electrode 150 can to be configured so that the electrodes are associated with the inner walls of the at least one chamber 120. In an embodiment of the present invention, the first electrode 140 and the second electrode 150 can to be configured as “fingers” that project from the inner walls of the at least one chamber 120. In an embodiment of the present invention, the electrodes configured as “fingers” may be interdigitated. In an embodiment of the present invention, the electrodes can be configured so that the first electrode 140 is associated with an inner wall of the at least one chamber and the second electrode 150 is configured as a “finger” that projects from an inner wall of the at least one chamber 120. In an exemplary embodiment of the present invention, the first electrode 140 and the second electrode 150 can be placed on opposite sides of the at least one chamber 120 containing the medium 160. (See FIG. 1). In an exemplary embodiment of the present invention, the first electrode 140 can be placed on the bottom of the at least one chamber 110 and the second electrode 150 can be placed on the top of the at least one chamber 110. (See FIGS. 2 A-C).

The electrodes of the ablation apparatus 110 are used to create an arc discharge within the chamber. When sufficiently high energy is supplied to the electrodes, a large field strength electric field is created thereby causing an arc discharge or other dielectric breakdown event to occur between the electrodes. This arc discharge is a highly self sustained discharge phenomenon of high current occurring between two closely spaced charged electrodes. In an embodiment of the present invention, this discharge leads to developing certain hot spot locations of high electric power densities on the electrodes that enables a phase change, producing conducting plasma between the electrodes. The produced plasma comprises electrons and ions of the electrode material and the medium 160 contained in the chamber, and the plasma is characterized by high energy content within the chamber. Once discharge of the arc occurs, the medium 160 within the chamber is subjected to a rapid, heat-induced volume expansion. As the volume of the medium 160 expands, the medium is ejected through the nozzle 130 of the at least one chamber 120 because of the high pressure generation within the chamber. The nozzle 130 facilitates the release of pressure within the at least one chamber 120 by accelerating the medium 160 residing in the chamber at high velocity out of the at least one chamber 120 onto the target surface. In the various embodiments of the present invention, the arc need not impact the target surface and there need not be current passage to or through the barrier. In an exemplary embodiment of the present invention, the electrical current does not contact the surface.

The creation of an arc discharge (e.g., a plasma discharge) depends strongly upon the distance between the electrodes. In an embodiment of the present invention, the proximity of the electrodes can be controlled by controlling the chamber layer thickness. In an embodiment of the present invention, the electrodes of the present invention are separated by about 1 μm to about 1 cm, or about 10 μm to about 100 mm, or about 100 μm to about 1 mm. In an exemplary embodiment of the present invention, the electrodes of the present invention are separated by about 250 μm.

A minimum amount of energy can be used for the creation of an arc discharge in the ablation system 100. The voltage that is supplied to the electrodes is determined based on the minimum power that is required to create an arc discharge between the electrodes, which would in turn depends on the spacing between the electrodes. Furthermore, the surface area of the electrodes can be varied depending on the volume of the device, the composition of the media, and the distance between the electrodes.

The electrical energy required to create an arc discharge for the ablation system 100 of the present invention could be provided by many electrical energy supply components, for example but not limited to discharge capacitors, a direct current (DC) power supply box, or a battery, among others. In an embodiment of the present invention, the electrical energy to create an arc discharge for the ablation system 100 is a power supply comprising discharge capacitors, resistors, and MOSFET switches. In this embodiment of the present invention, a voltage of 150 V can be supplied for a time span of 0.1-5 ms using a pulse generator, although voltages between about 1 V and about 10,000 V or about 10 V and about 1,000 V can be applied for time spans of about 10 ns to about 1 s or about 100 ns to about 100 ms or about 100 ns to about 10 ms. The voltage supplied to the capacitors from a high voltage DC power supply is stored as energy in the capacitors and later discharged to the electrodes by a pulse input from a pulse generator for the desired amount of time. The capacitance of the capacitor can comprise about 100 μF to about 600 μF. The voltage required to produce an arc discharge may comprise at least about 10 V. In an embodiment of the present invention, the voltage required to produce an arc discharge can comprise about 100 V to about 150 V, but could be much larger. In an embodiment of the present invention that utilizes a battery as the energy supply, an external circuit can be used to boost the voltage. For example, a voltage of about 7-11 V can be boosted to about 100-150 V in a very short period of time. This external circuit can comprise energy storage elements such as an inductor, capacitor, and other electrical components, including but not limited to a diode and MOSFET switches. One of ordinary skill in the art, however, would realize that the amount of energy (e.g., voltage) required to produce an arc discharge is inversely proportional to the distance between the electrodes.

In an embodiment of the present invention, an ablation device may comprise a unitary device comprising an energy supply component and an ablation system component. In another embodiment of the present invention, an ablation device may comprise a multi-component device comprising at least two separate components, an energy supply component, and an ablation system component The ablation device may further comprise, but is not limited to, microneedles, analyte sensing or retrieval components, fluid sampling components, cooling components, or transdermal active agent delivery components, formulations for delivery of active agents (e.g., a patch), each of which may be incorporated into either device, the unitary device or the multi-component device.

Using methods and apparatus of the present invention, the target surface for ablation is not electrically involved in the arc discharge process (i.e., no electrical current is passed to or through the surface (e.g., the skin)). Since arc discharge is a very fast process, with duration typically being in nanoseconds to microseconds, the skin is exposed to the ejected medium for a very short time, typically less than one (1) millisecond. In an embodiment of the present invention, ablation of a surface occurs in less than about 100 μs. In an embodiment of the present invention, ablation of a surface occurs in less than about 50 μs. In an embodiment of the present invention, ablation of a surface occurs in about 10 μs. In an embodiment of the present invention, ablation of a surface occurs in about 13 μs.

The dimensions of the nozzle 130 play an important role in controlling the size of the exposed tissue surface affected by the microfluidic jet. The area of the surface exposed or affected by discharge of a microfluidic jet can be controlled by controlling the size and shape of the nozzle opening. The nozzle 130 of the ablation apparatus 110 of the present invention can comprise many shapes, including but not limited to, a conical shape, a cylindrical shape, a cuboidal shape, or polygon shape, among others. In an embodiment of the present invention, the nozzle 130 can be tapered. The nozzle 130 may taper into the chamber. In an alternative embodiment, the nozzle 130 may taper out of the chamber. A tapered nozzle can comprise many shapes including but not limited to a conical shape, a cylindrical shape, a cuboidal shape, or polygon shape, among others. In an embodiment of the present invention, a nozzle 130 can have a radius of about 10 μm to about 1 cm or about 100 μm to about 1 mm. In an exemplary embodiment of the present invention, a nozzle 130 can have a radius of about 25 μm to about 200 μm.

In an embodiment of the present invention, a nozzle 130 may comprise an angled member, for example but not limited to an elbow. A nozzle 130 having an angled member may permit uncoupling of the thermal ablation process from mechanical components of the ablation process associated of the ablation system. More particularly, a nozzle 130 having an angled member would inhibit the mechanical effects of the microfluidic jet, including reducing the physical impact of the jet and any particulate matter of the medium with the target surface. Particulate matter could originate from the medium 160 or from the first electrode 140 or the second electrode 150. In an embodiment of the present invention, the nozzle 130 may be integrated in the at least one chamber 120 of the ablation apparatus 110. In an alternative embodiment of the present invention, the nozzle 130 can be fabricated as a separate and distinct structure from the at least one chamber 120.

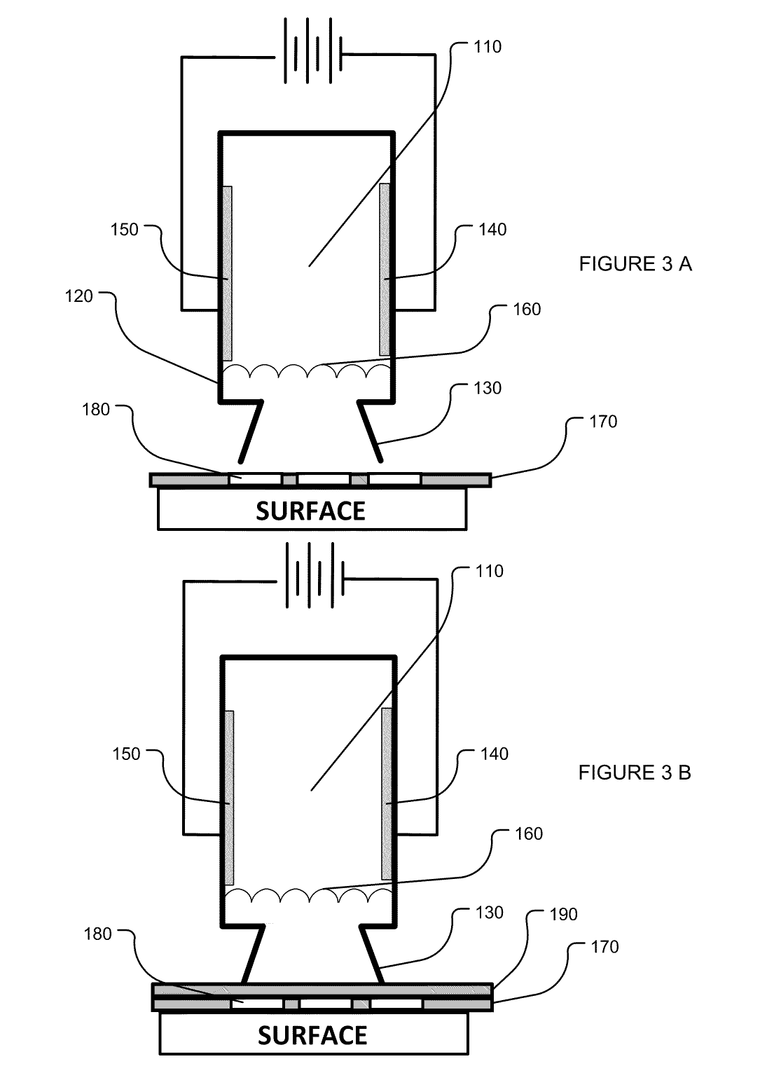

An aspect of the present invention comprises an ablation system 100 comprising: an ablation apparatus 110 comprising at least one chamber 120 having a nozzle 130, the at least one chamber 120 comprising a first electrode 140 and a second electrode 150, wherein the first electrode 140 and second electrode 150 are configured to permit the discharge of an arc therebetween; a medium 160 contained with the at least one chamber 120; and an interface layer 170. (See, for example FIGS. 3A-B). In an embodiment of the present invention, the interface layer 170 is provided between the ablation system 1020 and the target surface.

In an embodiment of the present invention, the interface layer 170 functions to further control the thermal and mechanical ablation of a surface. In an embodiment of the present invention, the interface layer 170 can comprise a layer of material. In an embodiment of the present invention, the layer of material possesses both thermally conductive properties and mechanical integrity, for example but not limited to metals, non-metals, ceramics, polymers, organics, inorganics, composites, or combinations thereof. Exemplary embodiments of an interface layer 170 comprise tungsten, titanium, and nickel, among others. In this embodiment of the present invention, the interface layer functions to uncouple the thermal ablative effects of the ablation apparatus 110 from the mechanical ablative effects of the apparatus. The interface layer 170 possesses sufficient mechanical integrity to inhibit mechanical force of the propelled medium; however, the thermal conductivity of the interface layer 170 permits the transfer of heat of the propelled medium to the surface covered by the interface layer.

In an embodiment of the present invention, the interface layer 170 comprises a plurality of holes 180. (FIG. 3A). The plurality of holes 180 may have a diameter of about 1 μm to about 1 cm, about 10 μm to about 1 mm. In an exemplary embodiment of the present invention, the plurality of holes 180 can have a diameter of about 100 μm. Thus, provided the diameter of the holes of the interface layer 170 are smaller than the diameter of the microfluidic jet emitted from the nozzle 130 of the at least one chamber 120, the interface layer 170 is capable of spatially controlling the thermal and mechanical ablative effects of the ablation apparatus 110. In such embodiments, the interface layer 170 may comprise materials that lack thermal conductivity but possess mechanical integrity, for example but not limited to metals, non-metals, ceramics, polymers, organics, inorganics, composites, or combinations thereof. Exemplary embodiments of a thermally insulative interface layer comprise polymeric materials, including but not limited to polydimethylsiloxane (PDMS) and Poly(methyl methacrylate) (PMMA), acrylics, plastics, including but not limited to Mylar and Kapton, combinations thereof, among others. Other materials, such as metals, can also be used as the thermally insulative interface layer, such as metals with relatively low thermal conductivity, including but not limited to titanium, and with sufficient thickness, such as greater than about 25 μm.

In addition to the composition of the interface layer, the thickness of the interface layer comprises another variable as thicker materials generally provide increased mechanical strength and decreased heat transfer across the material. Thus, the plurality of holes 180 of the interface layer 170 would permit thermal and mechanical ablation of the target surface, but the interface layer 170 would inhibit the transfer of heat and mechanical force of the propelled medium to the surface covered by the interface layer 170.

In an embodiment of the present invention, the interface layer comprises a mask 190. In an embodiment of the present invention, the mask 190 may comprise a mosaic of thermally conductive regions and thermally insulative regions, both of which possess mechanical integrity. (FIG. 3B). In this embodiment of the present invention, the interface layer 170 functions to uncouple the thermal ablative effects of the ablation apparatus 110 from the mechanical ablative effects of the apparatus. The interface layer 170 possesses sufficient mechanical integrity to inhibit mechanical force of the ejected medium. The mosaic of thermally conductive regions and thermally insulative regions permits the selective transfer of heat of the ejected medium to the surface covered by the thermally conductive regions, but inhibits the transfer of heat to the surface covered by the thermally insulative regions. In an exemplary embodiment of the present invention, thermally conductive regions and thermally insulative regions of the mask 190 can be comprise metal, non-metals, ceramics, polymers, organics, inorganics, composites or combinations thereof, where thermal conductivity or insulation is determined based on material properties, as well as material geometry, such as thickness. Exemplary embodiments of thermally conductive regions of an interface layer 170 comprise tungsten, titanium, and nickel, among others. Exemplary embodiments of thermally insulative regions of the interface layer 170 comprise polymeric materials, including but not limited to polydimethylsiloxane (PDMS) and Poly(methyl methacrylate) (PMMA), acrylics, plastics, including but not limited to Mylar and Kapton, and combinations thereof, among others. Other materials such as metals can also be used as the thermally insulative interface layer, especially using metals with relatively low thermal conductivity, such as titanium, and with sufficient thickness, such as greater than about 25 μm. In an embodiment of the present invention, the mask 190 may comprise the interface layer 170.

An aspect of the present invention comprises a method for ablating a surface comprising: providing to a surface an ablation apparatus comprising at least one chamber having a nozzle, the at least one chamber comprising a first electrode and a second electrode, wherein the first electrode and second electrode are configured to permit generate a current therebetween and a medium contained within the at least one chamber; generating a current between the first and second electrode; and ejecting the medium contained with the at least one chamber through the nozzle in the direction of the surface. The method for ablating a surface further comprises transferring the energy of the medium to a surface, wherein the energy of the medium is thermal energy. The method for ablating a surface further comprises transferring the energy of the medium to a surface by contacting the medium with a surface, wherein the energy of the medium is thermal energy and mechanical energy. In an embodiment of the present invention, generating a current comprises inducing an arc discharge.

An aspect of the present invention comprises a method for increasing the permeability of a barrier comprising: providing to a barrier an ablation apparatus comprising at least one chamber having a nozzle, the at least one chamber comprising a first electrode and a second electrode, wherein the first electrode and second electrode are configured to generate a current therebetween and a medium contained within the at least one chamber; generating a current between the first and second electrode; ejecting the medium contained with the at least one chamber through the nozzle in the direction of the barrier; and increasing the permeability of the barrier. In an embodiment of the present invention, generating a current comprises inducing an arc discharge.

The method for increasing the permeability of a barrier further comprises transferring the energy of the medium to a surface, wherein the energy of the medium is thermal energy. The method for increasing the permeability of a barrier can further comprise transferring the energy of the medium to a surface by contacting the medium with a surface prior to increasing the permeability of the barrier, wherein the energy of the medium is thermal energy and mechanical energy.

Aspects of methods of the present invention can increase the permeability of the barrier by many ablative mechanisms. The conditions necessary to achieve these mechanisms can be modulated to achieve an effective amount of barrier permeability. In one embodiment of the present invention, an ablative mechanism comprises the high velocity impact of the fluid jet with the barrier. The physical force of the impact of the microfluidic jet can damage the barrier to increase its permeability. In an embodiment of the present invention, an ablative mechanism comprises heat, as the heat of the medium can be transferred to the barrier. The thermal ablative effects on the barrier can alter the barrier structure to increase its permeability. In an embodiment of the present invention, an ablative mechanism can comprise the chemical effect of the medium on the barrier surface. In an embodiment of the present invention, chemicals can be applied to the surface prior to exposing the surface to thermal or mechanical ablative effects. In an embodiment of the present invention, chemicals can be added to the medium prior to initiation of the current (e.g., arc discharge). Chemicals, referred to as ablation materials, applied to the barrier can increase permeability of the barrier by dissolving, extracting, or otherwise altering the barrier permeability. Examples of such ablation materials include, but are not limited to, liquids, gels, solids, hydrophobic liquids, hydrophilic liquids, water, ethanol, methanol, organic compounds, alcohols, ketones, aldehydes, amines, ethers, esters, oils, paraffins, fatty acids, salt hydrates, including but not limited to calcium hydrates, sodium sulphate decahydrate, sodium phosphate dodecahydrate, calcium chloride hexahydrate, sodium thiosulfate pentahydrate, and mixtures or combinations thereof. In addition, combinations of thermal, mechanical, and chemical ablative mechanism can be used to increase the permeability of a barrier. By way of example, a microfluidic jet with high velocity and elevated temperature could have a combined mechanical and thermal ablative effect, forming holes in a target surface (e.g. the stratum corneum of the skin). Addition of chemical enhancers, such as ethanol, to the medium can add a chemical enhancement effect to the mechanical and/or thermal effect. In an embodiment of the present invention, increased permeability can be measured by increased electrical conductivity, increased transport of molecules across the barrier by diffusion or some other driving force, increased water loss from the tissue, or other methods known in the art.

An aspect of the present invention comprises an active agent delivery system comprising: an ablation apparatus comprising at least one chamber having a nozzle, the at least one chamber comprising a first electrode and a second electrode, wherein the first electrode and second electrode are configured to permit the discharge of an arc therebetween; a medium contained within the at least one chamber; and an active agent. In an embodiment of the present invention, the active agent may be associated with the medium contained in the at least one chamber.

An aspect of the present invention comprises a method of delivery of an active agent comprising: providing to a surface an ablation apparatus comprising at least one chamber having a nozzle, the at least one chamber comprising a first electrode and a second electrode, wherein the first electrode and second electrode are configured to permit the discharge of an arc therebetween and a medium contained within the at least one chamber; inducing an arc discharge between the first and second electrode; ejecting the medium contained with the at least one chamber through the nozzle in the direction of the surface; increasing the permeability of the surface; and delivering an active agent to or across the surface.

As used herein, “active agent” means a pharmaceutical or biotechnological compound or construct that induces a biological, pharmacological, or cosmetic effect on an organism. An active agent can be a compound, molecule, chemical, or biological construct that provides a physical or chemical change to an existing condition. An active agent can also be an analyte, as defined below.

The methods and apparatus of the present invention permit the delivery of active agents, including therapeutics, diagnostics, and prophylactics that may or may not be delivered using transdermal delivery systems currently known in the art. The delivery of many agents is limited by the barrier functions of skin or membranes of organisms. Active agents of the present invention include, but are not limited to: agents for gene therapy; nucleic acids; DNA; RNA; polynucleotides; peptides; proteins; amino acids; carbohydrates; viruses; antigens; immunogens; antibodies; chemical or biological materials or compounds that induce a desired biological or pharmacological effect; anti-infectives, such as antibiotics and antiviral agents; analgesics and analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including potassium and calcium channel blockers, beta-blockers, alpha-blockers, and antiarrhythmics; antihypertensives; diuretics and antidiuretics; vasodilators including general coronary, peripheral and cerebral; central nervous system stimulants; vasoconstrictors; cough and cold preparations, including decongestants; hormones, such as estradiol and other steroids, progesterone and derivatives, testosterone and derivatives; corticosteroids; angiogenic agents; antiangeogenic agents; hypnotics; immunosuppressives; muscle relaxants; parasympatholytics; nicotine; psychostimulants; sedatives; tranquilizers; ionized and nonionized active agents; cells; and compounds of either high or low molecular weight, among others. An active agent can further comprise a particle or plurality of particles, wherein a particle may induce a biological, pharmacological or cosmetic effect on an organism. Particles can comprise metals, non-metals, ceramics, polymers, organics, inorganics, composites, or combinations thereof. Examples of particles comprise, but are not limited to, liposomes, viruses, polymer particles that encapsulate active agents, which are released over time, coated particles that facilitate delivery of an active agent, wherein the particles comprise gold, polystyrene, glass, tungsten, platinum, ferrite, glass, or latex, among others. The active agents may have local effects, such as providing for a local anethesia, or may have systemic effects. The present invention is contemplates the mode of delivery of active agents, and is not limited by the particular active agents delivered. Other methods for increasing transport of molecules across skin or other membranes may be used with the present invention, such as microneedles, ultrasonication, electroporation, iontophoresis, electroosmosis, or convective fluid flow techniques.

An embodiment of the present invention comprises an active agent delivery system comprising an ablation apparatus 110 comprising at least one chamber 120 having a nozzle 130, the at least one chamber 120 comprising a first electrode 140 and a second electrode 150, wherein the first electrode 140 and second electrode 150 are configured to permit the discharge of an arc therebetween; a medium 160 contained with the at least one chamber 120; at least one active agent; and a formulation. In an embodiment of the present invention, the formulation comprises the at least one active agent. Formulations for transdermal and transmucosal drug delivery devices can be laminated composites that include a pressure-sensitive adhesive layer which may contain the active agent and by which the device is attached to the skin, and a backing layer, which forms the outer surface of the device, may form a reservoir for the active agent, and is impermeable to the active agent. Current transdermal patches are generally used to deliver certain types of molecules, such as those with low molecular weight (e.g., <500 Da) and octanol-water partition coefficients much greater than one. When used in combination with arc-based ablation, a much broader variety of active agents could be delivered, as discussed above. An active agent may be delivered to a surface following ablation of a surface. An active agent may be delivered to an ablated surface in many forms, including but not limited to, topical formulations. Topical formulations can comprise pharmaceutically acceptable formulations using formulation methods known to those of ordinary skill in known in the art, including but not limited to liquids, solids, semi-solids, gases, gels, dispersions, emulsions, ointments, salves, creams, pharmaceutical carrier(s) or excipient(s), mixtures, and combinations thereof.

In an embodiment of the present invention, the ablation apparatus comprises a microfabricated device that is small enough to be embedded on a transdermal patch. The ablation apparatus is an integral component of the transdermal patch, such that upon discharge of an arc in the at least one chamber and the ejection of a medium, the barrier in proximity of the ablation apparatus is ablated, and the active agent of the medium or transdermal patch enters the holes in the barrier, and transits the barrier, such as entering the human or animal through the holes that are formed.

Transdermal patches are well known in the art. The various embodiments of the present invention includes all forms of transdermal delivery of active agents comprising an incorporated ablation system including, but not limited to, transdermal devices, such as devices with a fill and seal laminate structures, peripheral adhesive laminate structures and solid state adhesive laminate structure, or devices with the active agent incorporated in the adhesive. As used herein, a patch functions in the same manner as a transdermal patch, but a patch can be used on many barriers to supply compositions, such as active agents to the barrier, but is not limited to epidermis or dermis of human or animal skin as the barrier, as may be understood for transdermal patch. Transdermal drug delivery is discussed in general in Cleary, G. W., “Transdermal Drug Delivery”, Cosmetics & Toiletries, Vol. 106, pgs. 97-109, 1991, which is incorporated herein by reference. Transdermal devices for the delivery of a wide variety of biologically active agents have been known for some time, and representative systems, which utilize rate controlling membranes and in-line adhesives, are disclosed in U.S. Pat. Nos. 3,598,122, 3,598,123, 3,742,951, 4,031,894, 4,144,317, 4,201,211, and 4,379,454 which are incorporated herein by reference. Such devices generally comprise an impermeable backing, a drug or active agent reservoir, a rate-controlling membrane, and a contact adhesive layer, which can be laminated or heat sealed together to produce a transdermal delivery device. U.S. Pat. Nos. 5,013,293; 5,312,325 and 5,372,579 disclose an electrolytic transdermal patch provided with a current oscillator for the periodic delivery of an active agent, and are herein incorporated by reference. Other methods for control of transport are taught in Smith et al. 1995, and Bronaugh et al., 1999. The driving force for transport may include gradients in concentration, chemical potential, pressure, osmotic pressure, voltage, and other gradients. Methods may include diffusion, osmosis, convection, electrophoresis, electrosmosis, convective dispersion, and other mechanisms known in the art. As shown herein, these and other transdermal delivery devices can incorporate one or more ablative systems for thermal and/or mechanical treatment of the skin to aid in increasing the transdermal flux rate of the active agent and reduce the barrier properties of the skin or other membranes.

Transmucosal patches and inserts are well known in the art. See Davis and Illum, Clin. Pharmacokinet., 42:1107-1128; U.S. Pat. Nos. 5,346,701, and 5,908,637 and U.S. Patent Application Publication No. 20050215475. The term “transmucosal” is intended to mean the passage of an active agent or analyte into, out of or through a mucosal membrane of a living organism. Transmucosal delivery comprises the delivery of an active agent to or across mucous membranes, including but not limited to oral, buccal, sublingual, ocular, nasal, pulmonary, gastrointestinal (e.g., stomach, small intestine, large intestine, rectal), urinary (e.g., urethra, bladder), and vaginal membranes, among others. Active agent formulations can be delivered to a mucous membrane absorption site by many means known in the art, including but not limited to, dropping or spraying from a bottle into the eye, nasal, buccal, or sublingual cavity; by aerosolizing from an inhaler into the nasal and pulmonary regions; as well as by applying a tablet, capsule, permeable matrix, soluble matrix, or other known dosage forms to the oral buccal, sublingual, ocular, nasal, pulmonary, gastrointestinal, urinary, and vaginal membranes.

An aspect of the present invention comprises a method of transdermal delivery of an active agent comprising: providing to the skin an ablation apparatus comprising at least one chamber having a nozzle, the at least one chamber comprising a first electrode and a second electrode, wherein the first electrode and second electrode are configured to permit the discharge of an arc therebetween and a medium contained within the at least one chamber; inducing an arc discharge between the first and second electrode; ejecting the medium contained with the at least one chamber through the nozzle in the direction of the skin; increasing the permeability of the skin; and delivering an active agent.

In an embodiment of the present invention, a method of transdermal delivery of an active agent comprises: providing to the skin an ablation apparatus comprising at least one chamber having a nozzle, the at least one chamber comprising a first electrode and a second electrode, wherein the first electrode and second electrode are configured to permit the discharge of an arc therebetween and a medium contained within the at least one chamber; inducing an arc discharge between the first and second electrode; ejecting the medium contained with the at least one chamber through the nozzle in the direction of the skin; increasing the permeability of the skin; removing the ablation apparatus; and delivering an active agent. In an embodiment of the present invention, the active agent can be delivered by applying a formulation, for example a patch, comprising the active agent. In this embodiment of the present invention, increasing skin permeability and the delivery of an active agent are a sequential process. The ablation of the skin is a pre-treatment, wherein the ablation apparatus functions to increase the permeability of the barrier surface. Thus, in this embodiment of the present invention, the medium need not have an active agent. Instead, the active agent is delivered to the permeabilized barrier after ablation of the barrier has occurred. An active agent may be delivered to a surface following ablation of a surface. An active agent may be delivered to an ablated surface in many forms, including but not limited to, topical formulations. Topical formulations can comprise pharmaceutically acceptable formulations using formulation methods known to those of ordinary skill in the art, including but not limited to liquids, solids, semi-solids, gases, gels, dispersions, emulsions, ointments, salves, creams, pharmaceutical carrier(s) or excipient(s), mixtures, and combinations thereof.

Methods of the present invention comprise increasing the transdermal flux rate of an active agent across a barrier, such as the skin or membranes of an organism, comprising using the devices described herein to increase the permeability of the barrier by applying an ablation system to a barrier, inducing an arc discharge between the first and second electrode; ejecting the medium contained with the at least one chamber through the nozzle in the direction of the barrier; increasing the permeability of the barrier without causing widespread damage to the barrier or underlying structures, reducing the barrier properties of the barrier to the transdermal flux rate of the active agent; while contacting the porated specific area with a composition comprising an effective amount of the active agent such that the transdermal flux rate of the active agent into the body is increased.

For example, methods of the present invention comprise increasing the transdermal flux rate of an active agent across the stratum corneum or epidermis of a human or animal, comprising providing an ablation apparatus comprising at least one chamber having a nozzle, the at least one chamber comprising a first electrode and a second electrode, wherein the first electrode and second electrode are configured to permit the discharge of an arc therebetween and a medium contained within the at least one chamber, the ablation system comprising a transdermal formulation, for example a patch, wherein one or more ablation apparatus are present on or in one surface of the transdermal formulation, such that application of the transdermal formulation to the stratum corneum of a human or animal brings the ablation apparatus in contact with the stratum corneum; inducing an arc discharge between the first and second electrode; ejecting the medium contained with the at least one chamber through the nozzle in the direction of the skin; increasing the permeability of the skin by forming one or more holes in specific sites in the stratum corneum without causing widespread damage to the stratum corneum, epidermis, or underlying dermal layers, reducing the barrier properties of the stratum corneum or epidermis to increase the transdermal flux rate of the active agent; and providing the porated stratum corneum or epidermis with a composition comprising an effective amount of the active agent such that the transdermal flux rate of the active agent into the body is increased.

As used herein, “transdermal flux rate” is the rate of passage of any active agent, including analytes, across the surface of the skin either into or out of the body. This term is commonly understood by those skilled in the art, and its usual and customary meaning is intended herein. Although the skin is used as an example, this invention is intended to include all barrier layers and flux rates across thereto, wherein skin is a representative example.

As used herein, “analyte” means any chemical or biological material or compound that may be measured, determined, monitored, and/or analyzed in order to gain information or determine the status related to the object or organism that is the source of the analyte. Examples of analytes include, but are not limited to, molecules and substances of diagnostic interest, natural and therapeutically introduced metabolites, hormones, amino acids, peptides and proteins, polynucleotides, cells, electrolytes, metal ions, suspected drugs of abuse, enzymes, tranquilizers, anesthetics, analgesics, anti-inflammatory agents, immunosuppressants, antimicrobials, muscle relaxants, sedatives, antipsychotic agents, antidepressants, antianxiety agents, small drug molecules, glucose, cholesterol, high density lipoproteins, low density lipoproteins, triglycerides, diglycerides, monoglycerides, bone alkaline phosphotase (BAP), prostate-Specific-Antigen (PSA), antigens, bilirubin, lactic acid, pyruvic acid, alcohols, fatty acids, glycols, thyroxine, estrogen, testosterone, progesterone, theobromine, galactose, urea, uric acid, alpha amylase, choline, L-lysine, sodium, potassium, copper, iron, magnesium, calcium, zinc, citrate, ammonia, lead, lithium, morphine, morphine sulfate, heroin, insulin, interferons, erythropoietin, fentanyl, cisapride, risperidone, infliximab, heparin, steroids, neomycin, nitrofurazone, betamethasone, clonidine, acetic acid, alkaloids, salicyclates, and acetaminophen.

An aspect of the present invention comprises a method of sampling an analyte contained by a barrier comprising: providing an ablation apparatus comprising at least one chamber having a nozzle, the at least one chamber comprising a first electrode and a second electrode, wherein the first electrode and second electrode are configured to permit the discharge of an arc therebetween and a medium contained within the at least one chamber; inducing an arc discharge between the first and second electrode; ejecting the medium contained with the at least one chamber through the nozzle in the direction of the barrier; increasing the permeability of the barrier; and sampling at least one analyte contained by the barrier. Various embodiments of the present invention provide for the extraction of analyte across the barrier, for example but not limited to non-invasive sensing of analytes in the bodily fluids.

Methods of the present invention comprise ablating a barrier with the apparatus described herein such that interstitial fluid, other bodily fluids, or fluids retained by the barrier, transport from or form in the holes; collecting or sampling the fluid in the hole; and analyzing, measuring or detecting, one or more analytes in the collected fluid. Collecting or sampling devices include, but are not limited to, a vacuum or absorption member that may be applied to the microporated area to remove the fluids. Alternatively, the fluid and any analytes therein may be analyzed, measured, or detected in situ in the hole, without removal of the fluids. Methods for determining or measuring analytes are known in the art, and include, but are not limited to colorimetric assays, immunoassays, specific binding partner assays, and other tests for analytes.

The methods of the present invention contemplate that the ablation system may be applied, stuck by adhesives, attached, bound, wrapped within a dressing, or by other means of attaching the component for a limited time period to a barrier. For example a transdermal patch ablation system may be applied the skin or membranes of a human or animal for about 0.5 minutes to 24 hours, for 1-6 days, or for weeks or months at a time. Other ablation systems may be applied to a barrier for longer periods, depending on the intended uses. The ablation systems of the present invention may be activated once to provide surface ablation to specific sites on the barrier or may be activated multiple times, including from 1 to 1000 times, from 1 to 20 times, from 5 to 50 times, and all times in between. The ablation system may remain in the same site on the barrier or may be moved to different sites, depending on the intended use.

The present invention contemplates the single use of ablation system followed by its disposal It must be noted that, as used in this specification and the appended claims, the singular forms “a”, “an”, and “the” include plural referents unless the context clearly dictates otherwise.

All patents, patent applications and references included herein are specifically incorporated by reference in their entireties.

It should be understood, of course, that the foregoing relates only to exemplary embodiments of the present invention and that numerous modifications or alterations may be made therein without departing from the spirit and the scope of the invention as set forth in this disclosure.

Although the exemplary embodiments of the present invention are provided herein, the present invention is not limited to these embodiments. There are numerous modifications or alterations that may suggest themselves to those skilled in the art.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

Therefore, while embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

### EXAMPLES

### Example 1

### The Effects of Millisecond- to Second-Long Thermal Exposures without a Mechanical Component

In order to understand the effects of thermal exposure on skin permeability, skin was exposed to heat sources ranging in temperature from 25 to 350° C. for durations of 0.1 to 5 s. Temperature was controlled by using either a hot plate or a soldering iron, each of which included controllers that maintained these instruments at pre-set temperatures. Exposure time was controlled by using an electronic solenoid device that pressed (e.g., contacted) the skin against the heat source for the desired time.

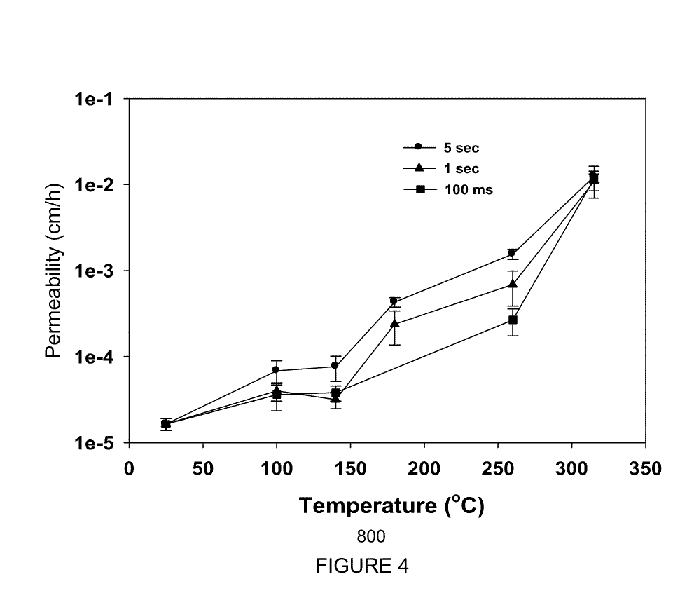

As shown in FIG. 4, skin permeability to a model drug, calcein, increased as a strong function of temperature and a weaker function of exposure time over the range of conditions studied. Over the temperature range of 25-150° C., skin permeability increased by a few fold. Additional tests using differential scanning calorimetry, thermogravimetric analysis and other tests suggested that the increase in permeability over this temperature range is due to reorganization of lipid bilayer structures in the stratum corneum and possible protein denaturation.

Over the temperature range of 150-250° C., skin permeability was increased by up to 100 fold, which should be significant for transdermal drug delivery applications. At 315° C., skin permeability was increased by 1000 fold, which represents a huge increase in the rate of transdermal transport. These large increases in skin permeability were associated with loss of stratum corneum mass as determined by thermogravimetric analysis, which were interpreted as chemical decomposition of the stratum corneum, perhaps by combustion.

Thermal exposure time was also an important factor, where longer exposures generally had larger effects on skin permeability. However, exposure time was not as important a factor as temperature over the 0.1-5 s exposure times considered.

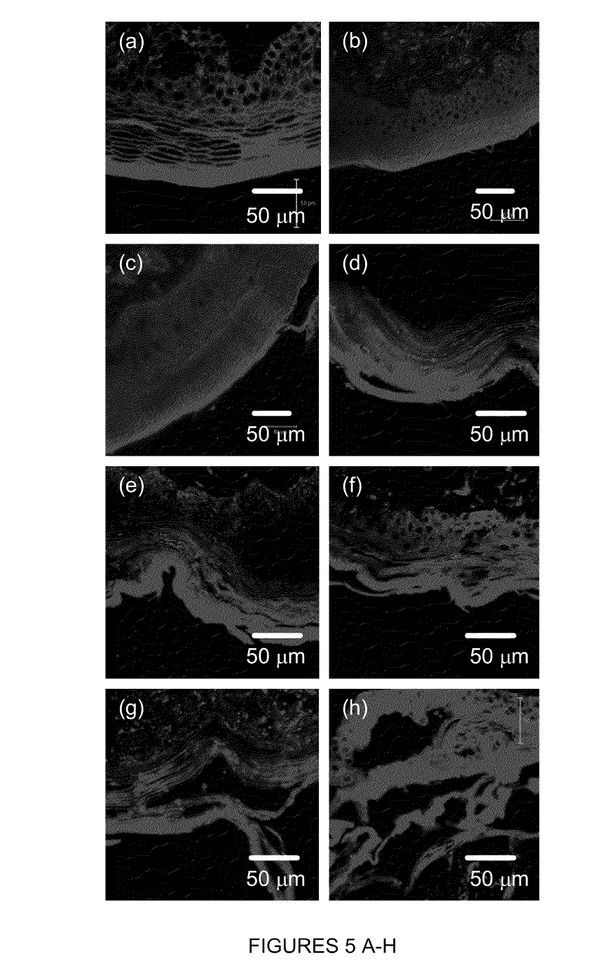

Despite these large increases in skin permeability, visual examination of the skin did not show noticeable effects. Microscopic analysis, however, did show micron-scale rearrangements of stratum corneum structure that are consistent with the data. After thermal treatment over a range of temperatures, skin was cryo-sectioned and imaged by confocal microscopy, as shown in FIG. 5. FIGS. 5 A-H provide confocal microscopy images of histological sections of stratum corneum stained with Nile Red after thermal exposure for 1 s at different temperatures: (a) control, (b) 100° C., (c) 140° C., (d) 160° C., (e) 180° C., (f) 200° C., (g) 260° C., and (h) 315° C. In the control sample (FIG. 5 A), the stratum corneum can be seen in the lower half of the image as red-stained extracellular lipid and oblong, unstained keratinocytes and the viable epidermis is seen in the upper half of the image as round cells with red-stained plasma membranes. As temperature is increased, stratum corneum structure becomes disrupted. For example, at 160-200° C. (FIGS. 5 D-F), stratum corneum structure appears disorganized but nonetheless intact overall, where as at 260-315° C., the stratum corneum structure is in significant disarray (FIGS. 5 G-H). This correlates with the permeability measurements, in which significant increases in skin permeability were observed above 150° C. and the very large increases were observed above 250° C.

### Example 2

### Wireless Thermal Ablation of Stratum Corneum

There are a variety of advantages to having a power source that need not be physically connected to the transdermal delivery patch. To generate thermal pulses to ablate the skin in this way, a wireless induction heating system was developed for generating micron-scale pores in the skin by thermal micro-ablation that seeks to combine the efficacy of previous wired approaches with the improved convenience and likely higher patient compliance of wireless power delivery. The separation between the power source and the heating elements provides the potential for design flexibility, such as easier integration of ablation heating components with the drug patches and removal of the inconvenience of being ‘plugged in’ to an energy source, while maintaining the advantages of thermal micro-ablation.

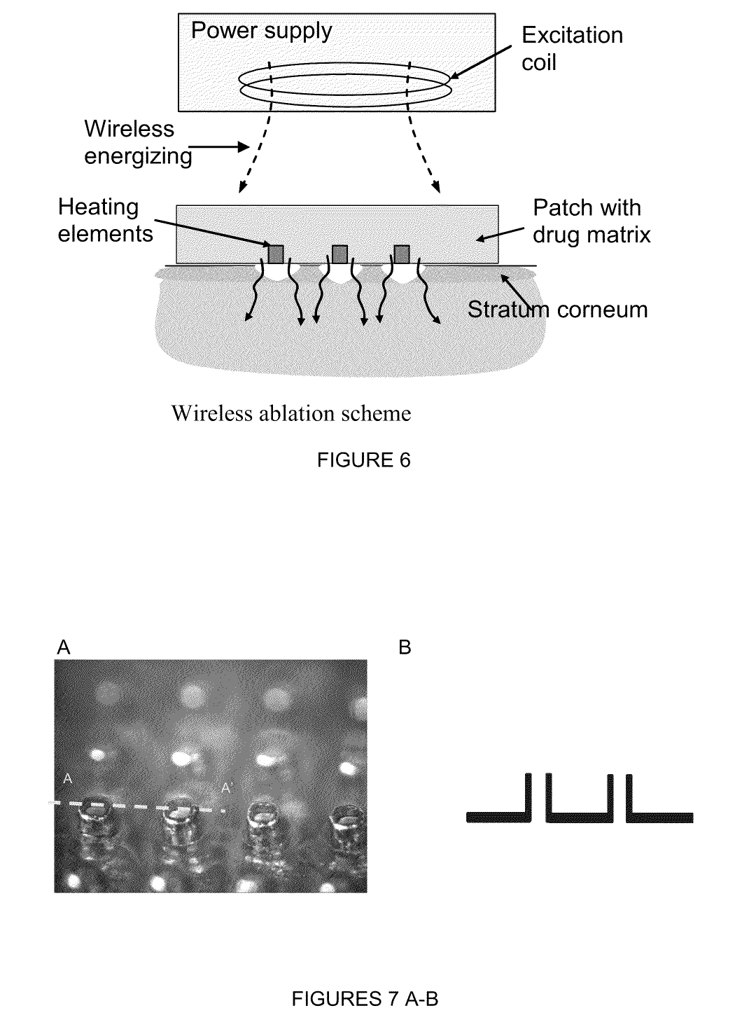

FIG. 6 shows a schematic diagram of the inductive heating system, including an AC power source with an excitation (induction) coil, which could be designed to be handheld, and micro-heating elements that could be integrated into a patch. The induction heating is based on eddy current and hysteresis loss induced in the heating elements by the alternating magnetic field of the excitation coil. In most metals, eddy current loss is the dominant source of induction heating. When a conductive material experiences alternating magnetic flux inside it, an electromotive force is induced in the material that causes a circulating current or eddy current, in accordance with Faraday's Law of Induction. This eddy current is converted into heat due to the Joule effect (i.e., resistive loss) in the heating material.

In the present design, the micro-heating elements consist of two functional materials: metal (nickel) and polymer (PDMS). Nickel was chosen as a heating material because it is nontoxic (although can be irritating to sensitized individuals) and has a high relative magnetic permeability that is favorable for induction heating. As shown in FIGS. 7 A-B, the heating material was structured to be a 20×20 array of hollow posts and a base plate. The base plate has at least two functions: one is to connect the array of hollow posts physically, and the other is to generate the induction (eddy current) heat and transfer to the hollow posts for rapid heating of the contacted skin. The PDMS layer is placed on top of the base plate to provide thermal insulation between the base plate and the skin. Therefore, in this design, only the end tip of the array of hollow posts and the PDMS layer will be contacted to the skin, and thermal ablation of skin would be localized to the shape of the post tip.

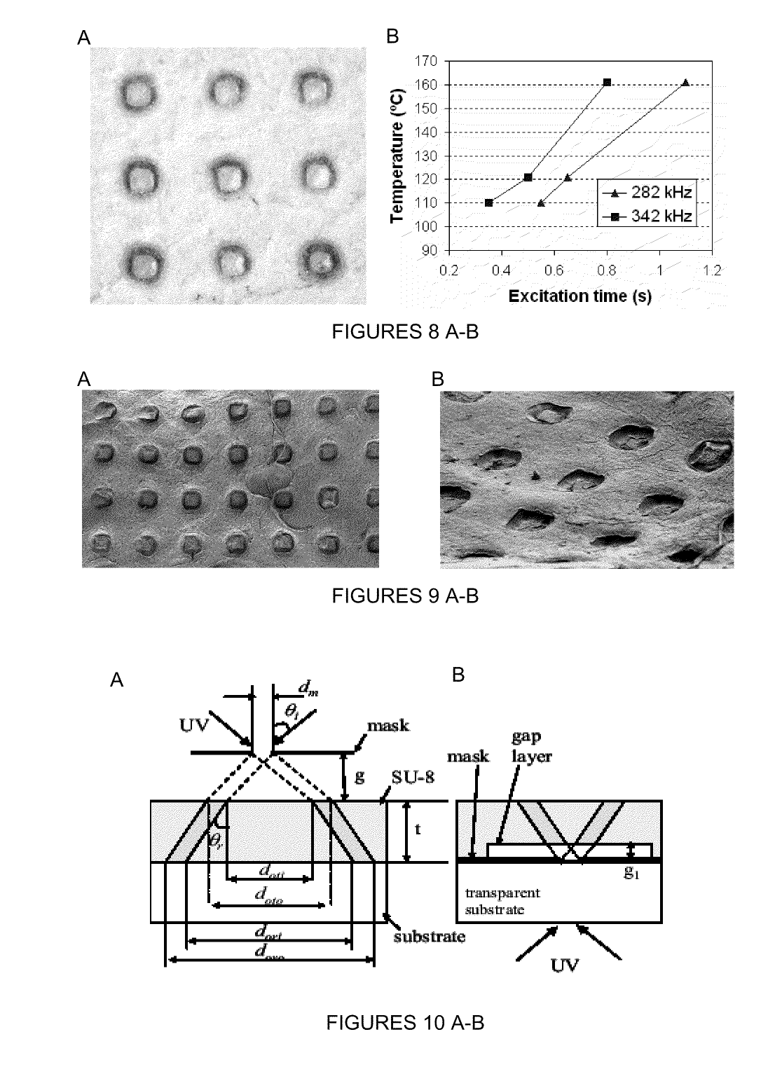

The induction heating performance of the fabricated hollow-post array has been characterized while applying an AC magnetic field with the excitation coil. As a simple “thermometer” that gives spatial information about temperature, liquid-crystal polymer (LCP) paper, which changes its color permanently when a temperature exceeds pre-set temperatures of 110, 121, or 161° C., was used as a temperature indicator for initial bench studies. FIG. 8A shows an example of the temperature-indicating paper after induction heater excitation. The paper clearly shows the localized heat pattern representative of the ring-shaped tip of the posts.

The hollow post array was placed on top of the temperature-indicator papers and an AC current of controlled duration (in time increments of 0.05 s) and specified frequency was applied to the coil. The resulting temperature data is shown in FIG. 8B. The excitation time was recorded when each LCP paper changed color. Therefore, the x axis of the graph represents the minimum time required to achieve the given temperature (shown on the y axis). The RMS magnetic field applied to the heating element was approximately 50 Gauss at frequencies of 282 and 342 kHz. Since eddy current loss in the micro-heating element increases with applied frequency, the higher frequency excitation produced higher temperatures than the lower frequency, as expected.

To assess the performance of the inductive heating system to ablate skin, the micro-heating elements were applied to human cadaver skin in vitro. FIG. 9 shows scanning electron micrograph (SEM) images of the skin (stratum corneum and epidermis) after the micro-heating elements were activated and removed. Sites of local skin micro-ablation in the position of an array of donut-shaped openings having the geometry of the tips of the hollow posts are evident. This figure indicates that fabricated micro-heating elements are able to generate localized micro-ablation in human skin.

### Example 3

### The Effects Millisecond-Long Microjet Exposures with Thermal and Mechanical Components Using Integrated Micro-Heater Devices

Having investigated purely thermal exposures and developed micro-heater arrays, the next experimental step was to design micro-nozzles to attach to the heaters, such that a liquid (or gel) formulation can be placed within the micro-nozzle reservoirs in contact with the micro-heaters. Upon actuation of the heaters, the liquid formulation can be vaporized and expelled through the micro-nozzles at the skin. In this way, the resulting hot microjet can impact the skin with thermal and mechanical components.

Toward this goal, arrays of multiple hollow nozzles suitable for jet ejection were fabricated using the technique of proximity-mode inclined UV lithography. Proximity mode inclined UV lithography is a new fabrication approach that we developed to enable the single-mask realization of solid and hollow three-dimensional microstructures of unusual shapes. Expanding upon previous inclined exposure approaches, a defined gap between the photomask and the substrate adds an additional degree of freedom to generate different ray trace patterns in the photoresist layer. The proximity approach can be used with both frontside and backside exposure approaches: the air gap is controlled by spacers with different thicknesses between the photomask and the substrate for front-side exposure, while UV transparent glass of known thickness on the substrate prior to photoresist deposition enables proximity reverse-side exposure. A horn shape has been achieved by reverse-side inclined exposure using the same photomask patterns used in nozzle fabrication.

Two types of proximity modes are illustrated in FIG. 10. FIG. 10 A shows an air gap inserted between the mask and the substrate for front-side exposure. The gap can be controlled by placing spacers of known thickness. A ray trace through a clear window with a diameter of dₘ in the optical mask can generate a revolving 3-D nozzle latent pattern in the SU-8 layer after inclined rotational exposure, while its geometrical dimensions can be determined by the incident angle θᵢ, the refractive index of SU-8 n_{SU-8}(≈1.67), the thickness of the photoresist layer t, and the gap between the photomask and the substrate g, and are described as follows:

dₒₜᵢ: inner diameter of the orifice tip=2*g/tan θᵢ−dₘ (1)

dₒₜₒ: outer diameter of orifice tip=2*g/tan θᵢ+dₘ (2)

dₒᵣᵢ: inner diameter of orifice root=2*g/tan θᵢ−dₘ+2*t*tan θᵣ (3)

dₒᵣₒ: outer diameter of orifice root=2*g/tan θᵢ+dₘ+2*t*tan θᵣ (4)

θᵣ: refracted angle=sin⁻¹(sin θᵢ*nₐᵢᵣ/n_{SU-8}) (5)

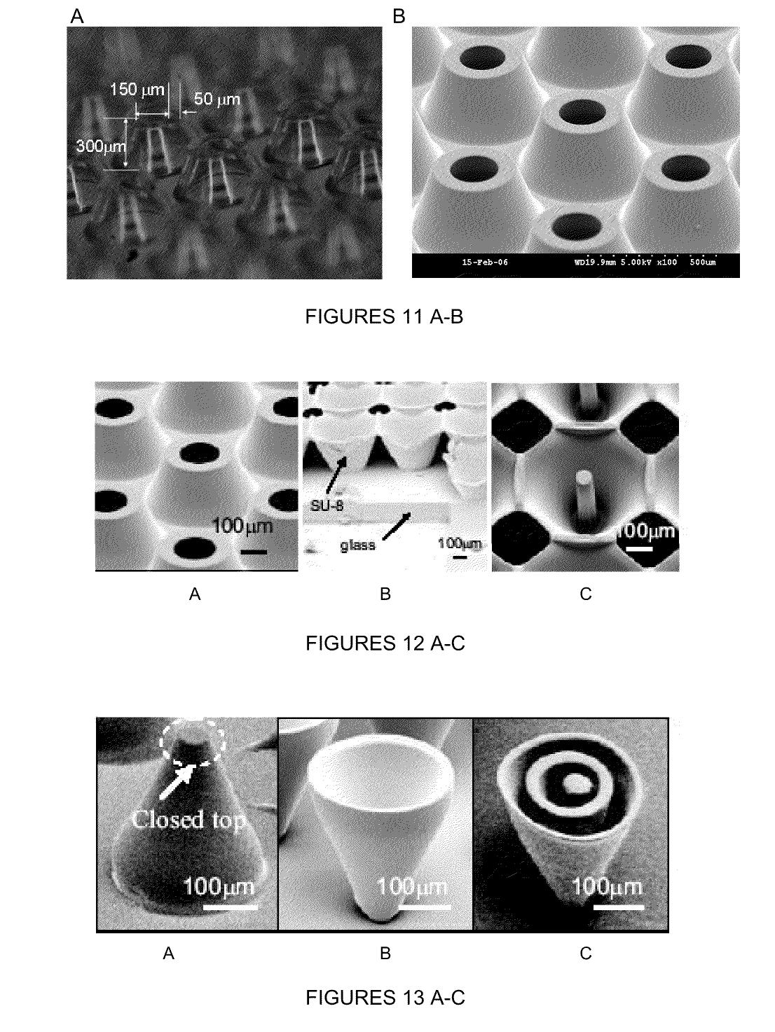

Proximity patterning can be implemented for reverse-side exposure by adding a known-thickness gap layer prior to SU-8 deposition as shown in FIG. 10 B, where both the substrate and the gap layer are UV transparent (e.g., glass). However the gap layer is not limited to glass but can be a UV-transparent polymer or ceramic. The substrate has a pre-patterned metal layer for a photomask, having a clear open window. The resultant pattern after reverse-side inclined rotational exposure will produce a horn shape. FIGS. 11 A-B show structures fabricated from a photomask with 50 μm diameter clear window patterns. The sections of the micro-nozzle arrays are shown by optical imaging (FIG. 11 A) and electron microscopy (FIG. 11 B).

More particularly, large areas of multiple hollow nozzles suitable for jet ejection can be fabricated using the technique of proximity-mode inclined UV lithography. Proximity mode inclined UV lithography is a fabrication approach enabling the single-mask realization of solid and hollow three-dimensional microstructures of unusual shapes. Expanding upon previous inclined exposure approaches, a defined gap between the photomask and the substrate adds an additional degree of freedom to generate different ray trace patterns in the photoresist layer. The proximity approach can be used with both frontside and backside exposure approaches: the air gap is controlled by spacers with different thicknesses between the photomask and the substrate for front-side exposure, while UV transparent glass of known thickness on the substrate prior to photoresist deposition enables proximity reverse-side exposure. With continuously varying air gap spacing, nozzles with various orifice sizes of 0 μm to 255 μm, a height of 250 μm, a side wall tilting angle of 25°, a wall thickness of approximately 60 μm have been successfully fabricated using front-side exposure with an incident angle of 45° and 50 μm-diameter of clear circle mask patterns. A horn shape has been achieved by reverse-side inclined exposure using the same photomask patterns used in nozzle fabrication.

Placement of photomasks in proximity to, rather than in contact with, the substrate has been widely used in standard UV lithography to prevent contamination or damage of the mask or the substrate and for photoresist patterning on an uneven substrate. In conventional proximity patterning, since the UV source is incident normal to the substrate, the transferred patterns follow the photomask image in shape, potentially with reduced resolution due to light diffraction at the edge of the pattern.

Recently, advanced UV lithography processes using SU-8, such as inclined exposure and reverse-side exposure, have been reported for complex three-dimensional (3-D) fabrication. When the inclined exposure technique is utilized in a rotational fashion, e.g. the substrate stage moving during exposure, various revolving patterns can be produced.

The inclined rotational exposure process has been further advanced by exploiting the proximity scheme to generate unusual 3-D patterns, which are different from the original mask patterns. Since the proximity gap between the mask and the photoresist plays an essential role to determine the resultant 3-D image, the gap effects for 3-D patterning have been investigated. To demonstrate its versatility, tapered micronozzles and conical microhorns have been fabricated from front-side exposure and reverse-side exposure, respectively. Mathematical equations for resultant dimension as a function of gap and incident angle have been provided and compared with the fabricated results.

FIG. 12 shows structures fabricated from the same photomask with 50 μm diameter clear window patterns: (a) a nozzle from front-side exposure, (b) a horn array with the lower portion truncated by the gap layer from reverse-side exposure, (c) a horn with a central column fabricated from additional vertical exposure after reverse-side inclined rotational exposure using the same mask.

As a reference, the contact mode structures are shown in FIG. 13: (a) a closed top conical shape from front exposure, (b) a horn from reverse-side exposure, and (c) a multi-layer horn from reverse-side exposure. The tips of the microhoms are as large as the mask layer.

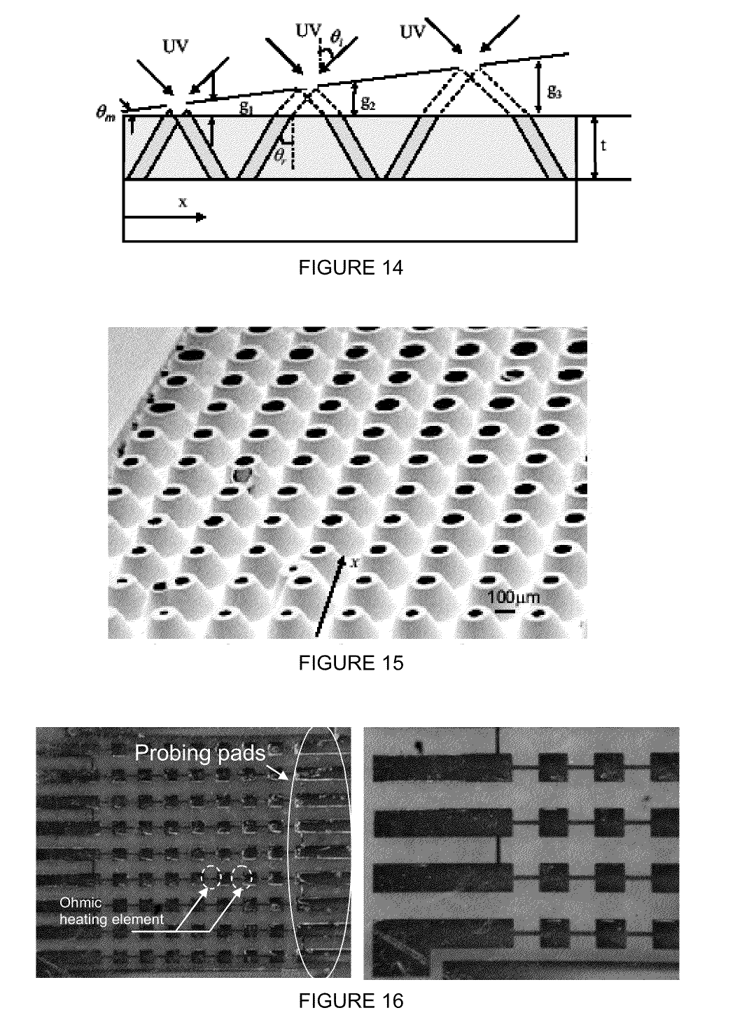

By implementing continuously varying air gaps between the photomask and the substrate, a micronozzle array with different orifice sizes can be simultaneously formed as shown in FIG. 14. The gap of the leftmost side is set to zero and that of the rightmost side has a spacer with a thickness of approximately 500 μm. The gap g is a function of the distance x from the leftmost side. Since the mask width is 1″ (equal to 25.4 mm), the gap is described as a function of the distance as following.

g=0.5x/25.4≈0.02x (6)

The mask tilting angle θₘ is approximately 2°, and therefore, the overall nozzle shape is not noticeably asymmetric due to the tilting gap. FIG. 15 shows a fabricated micronozzle array: (a) a gap g₁=25 μm, g₂=135 μm, and g₃=225 μm. The inner and outer orifice diameters of the fabricated nozzles are calculated using Eq. (1) and (2), respectively and show good agreement with the measurement results.

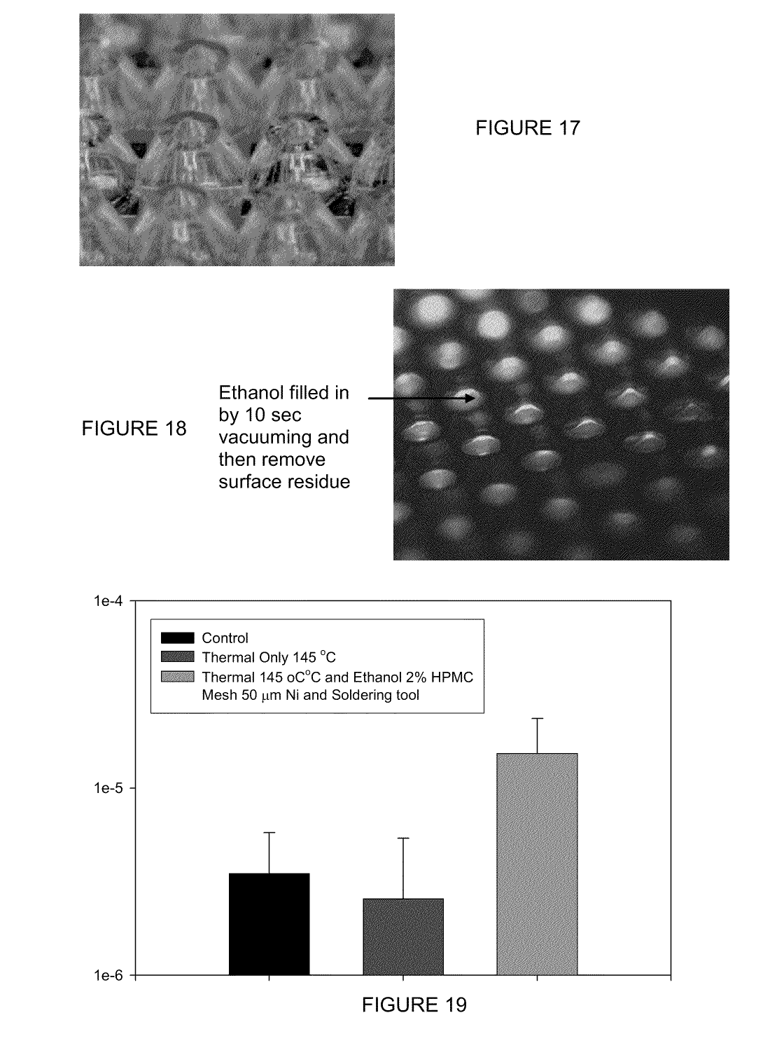

The heaters developed for induction-heating ablation of skin (FIG. 7) could be used in combination with the micro-nozzles to generate microjets. However, their micro-heater structure was designed for localized thermal contact with skin and is more complex than is needed for microjet formation. Therefore, a simpler micro-heater array was designed to combine with micro-nozzles for an integrated microdevice. To fabricate these micro-heaters, Pt (1000 Å) was deposited and patterned using standard lift-off processes. Next, SiO₂ (4000 Å) was deposited using plasma-enhanced chemical vapor deposition (PECVD). Then, Au (2000 Å) was deposited to create a pad that helps create a more even distribution of heat. This gold was patterned using the lift-off process. The SiO₂ for the probe pad was etched in buffered oxide etchant (BOE) and an Al layer (3000 Å), as subsequently deposited using E-beam evaporation before removing the photoresist used as the SiO₂ etch mask. The resulting microheater array is shown in FIG. 16. FIG. 17 shows an integrated device, in which the micro-nozzles have been bonded to the micro-heater array to form a micro-ablation system device.

This micro-ablation system was designed to cause mechanically-induced skin ablation at mildly elevated temperature. As such, the nozzle reservoirs of the micro-heater system were filled with ethanol because ethanol has a relatively low boiling point at 78° C. In this way, the micro-heaters could heat the ethanol until its boiling point. Then, the huge volume increase associated with vaporization would expel the ethanol vapor, and possibly some entrained ethanol liquid, at the skin with high velocity.

Considering that ethanol is a volatile solvent that would evaporate quickly during storage, ethanol was mixed with 1-4% hydroxyl-propyl-methyl cellulose (HPMC), which served as a thickening or gelling agent. This viscous ethanol solution was filled into reservoirs of the micro-nozzles by placing under vacuum for 10 sec. after which the surface residue was wiped off the surface. FIG. 18 shows the micro-ablation device filled with viscous ethanol gel.

As a first test of the hypothesis that rapidly heated ethanol can increase skin permeability, ethanol gel was filled into an array of micro-cavities and placed onto the skin. The backside of the ethanol-filled device was contacted with a 145° C. soldering iron tip for 1 sec. As shown in FIG. 19, this resulted in a 2-3 fold increase in skin permeability, which confirmed the hypothesis that rapidly vaporized ethanol can increase skin permeability. As a negative control, the 145° C. soldering iron tip was contacted to the back side of the device without ethanol, which was found to have no effect. This showed that the increased skin permeability was due to the hot ethanol ejectate and not due to heat alone.

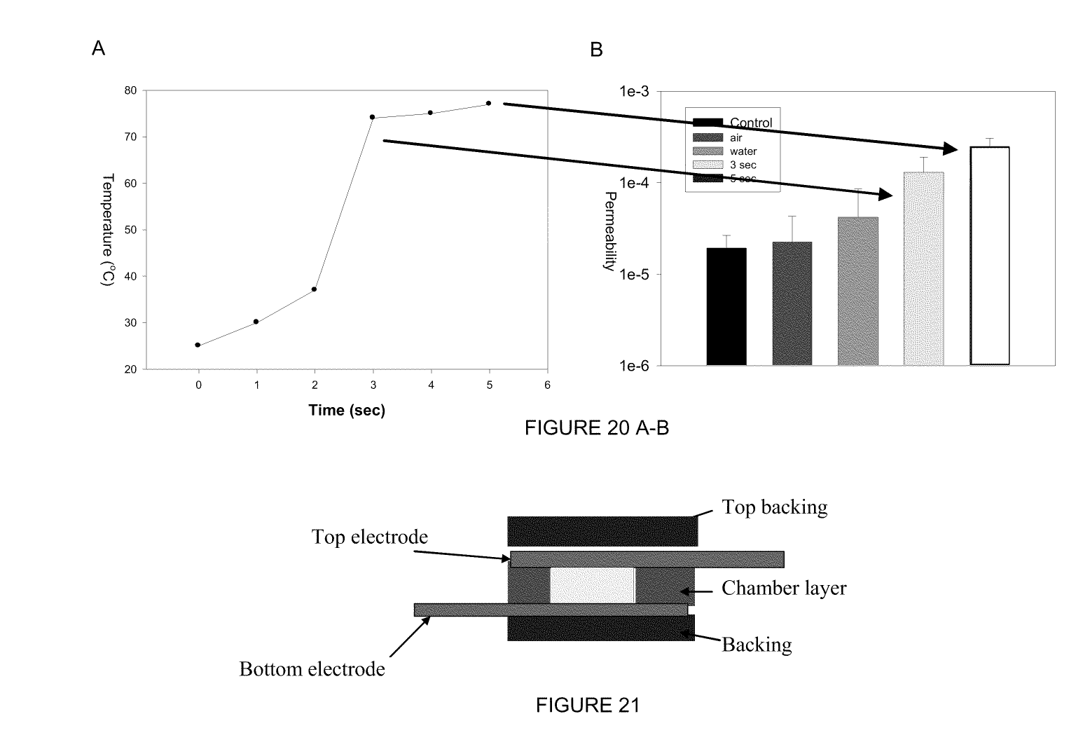

Guided by the preliminary data in FIG. 19, the micro-ablation system shown in FIGS. 11 A-B and 16-18 was used to assess its ability to increase skin permeability using millisecond-long exposure to moderate temperature (<100° C.) ethanol micro-jets. FIG. 20B depicts human cadaver skin permeability to calcein measured for intact skin (black bar), after micro-device heating for 3 sec with micro-reservoirs filled with air (dark gray bar), water (medium gray bar) or ethanol gel (light gray bar) or after micro-heater device heating for 5 sec with micro-reservoirs filled with ethanol gel (white bar). As shown in FIG. 20B, this approach increased skin permeability by as much as 10 fold. A negative control experiment, in which the micro-heaters were activated for 3 sec, but the micro-reservoirs were filled only with air (i.e., no ethanol), had no effect on skin permeability, which indicated that direct heating of the skin by the micro-heaters did not occur. Another negative control experiment, in which the micro-reservoirs were filled with water and heated for 3 sec increased skin permeability by 2-3 fold. However, filling the micro-reservoirs with ethanol gel and heating for 3 sec increased skin permeability by 5 fold. Heating with ethanol for 5 sec increase skin permeability by 10 fold.

FIG. 20 A illustrates the temperature inside the micro-reservoir filled with ethanol gel was measured as a function of time. The graph of FIG. 20A provides information about the temperature achieved inside the micro-reservoirs filled with ethanol gel. For the first 2 sec, temperature rose to approximately 37° C. This slow rise in temperature was probably due to a thermal lag time for heat to be generated within the heaters and to be transferred to the ethanol gel. By 3 sec, the micro-reservoir temperature surged to about 75° C., which is probably indistinguishable from the boiling point of ethanol at 78° C., given experimental uncertainty. From 3-5 sec, the temperature was relatively constant, although a small increase was measured. This is probably because it took 3 sec to heat the ethanol to its boiling point and then the temperature remained at the boiling point while ethanol boiled and was ejected from the micro-reservoir.

These temperature measurements help explain the skin permeability measurements. After 3 sec, ethanol had begun to boil and therefore eject from the micro-reservoir. This increased skin permeability. After 5 sec, even more ethanol had boiled and therefore increased skin permeability further. In contrast, 3 sec of heating may not have boiled much water, since water has a larger heat capacity and a higher boiling point than ethanol, which can explain why filling the micro-reservoirs with water was less effective.

### Example 4

### The Effects of Microsecond-Long Microjet Exposures With Thermal and Mechanical Components Using Arc-Discharge Microdevices

The previous experiments demonstrated that ethanol vapor microjets can significantly increase skin permeability. However, a better device design could improve upon the 10-fold increase observed. In the present design, the ethanol vaporization was occurring too slowly, which resulted in a microjet without sufficient velocity. To address this issue, the micro-device was re-designed so that it could heat much more rapidly. Rather than heating a heater, which then transferred heat to the ejectate medium formulation, heat was generated directly within the ejectate medium formulation by passing current through the ejectate medium. This was effectively accomplished by generating an arc across closely spaced electrodes by applying a high voltage pulse. In this context, the constraint that microjet temperature needed to be less than 100° C. was relaxed and therefore water was selected to fill the micro-reservoirs.

This ablation technique is based on the hypothesis that microsecond-long, high-temperature microjets can selectively remove stratum corneum to increase skin permeability by orders of magnitude. This approach differs from other thermal ablation methods that use millisecond pulses, which cannot localize heating to the stratum corneum as efficiently as the microsecond pulses used here and thereby risk damaging deeper tissue.

Microjets were formed in this study by designing and fabricating microdevices that generate an electrical arc across closely spaced electrodes, which ejects a droplet of vaporized water at the skin within 100 μs. The impact of this high-temperature, high-velocity microjet is sufficiently transient that it has highly localized effects to the stratum corneum, but nonetheless can increase skin permeability by orders of magnitude. Moreover, this microdevice design is suitable for low-cost mass production.

In fabricating these microjets, micromachining approaches were considered with an emphasis placed on utilizing the simplest fabrication schemes available. Also, as these microjets ejectors can be one-time-use devices, fabrication techniques that enable easy batch fabrication and concomitant ease of high volume manufacturing were considered. The devices were fabricated using various micromachining techniques that enable patterning and etching of sub-micron geometries in a variety of materials. In the present study, laser micromachining techniques and lamination of low-cost polymers and metals were used for fabricating different components of the microjet ejector. FIGS. 2 B-C show a schematic of a single arc-discharge jet ejector fabricated by laser micromachining. Although devices made from laser processing and lamination techniques were used and discussed in this study, other machining techniques, such as lithography and molding could be used for the device fabrication. Also, low cost plastics, metals, and polymeric materials which make it affordable to produce these devices in a large scale were used in fabricating different components of the micro jet ejector.

In an embodiment of the present invention, the microjet ejector assembly has four components: a chamber, two electrodes and a nozzle. The chamber houses fluid to be ejected, typically an aqueous solution containing a drug model, salt, gelling agent, and optional gold particles, while the electrodes were used to create an arc discharge within the chamber.

The chamber and the substrate layers are patterned in a Mylar layer, which is a low cost polymer, using a CO₂ laser that has a spatial micromachining resolution of 100 μm. The thickness of the chamber layer is 250 μm. The electrodes were made by patterning an inexpensive thin metal film such as brass or nickel using an IR laser. Feature sizes as small as 60 μm can be machined by the IR laser. The thickness of the metal used is about 25 μm to about 50 μm. Conical or cylindrical nozzles are fabricated either by integrating these along the chamber in the same layer or are fabricated as a separate layer. These layers are then adhered together and laminated to the substrate layer in a hydraulic press between aluminum molds.

The lamination sequence comprises the following steps: 1) laminate the bottom electrode onto the base substrate, which helps provide the mechanical strength to the electrode layer, thus reducing any deformations caused due to mechanical or thermal effects during operation; 2) the chamber and nozzle layers are then laminated on top of the bottom electrode and the chamber is filled with the desired solution; and 3) the filled chambers are sealed by laminating with a top electrode and a supporting backing layer.

Both individual devices as well as arrays were fabricated. As the creation of arc discharge depends strongly on the distance between the electrodes, optimum chamber thickness is chosen based on this distance. Nozzles with diameter ranging from 50 to 400 μm and chambers with volume ranging from 1-8 mm³ with distance between electrodes of 250 μm were considered. The device was actuated by applying a charged capacitor to the device electrodes through a MOSFET switch and, upon triggering of the switch, discharging the capacitor through the ejectate formulation via the electrodes. Capacitances varying between 100-600 μF and voltages of 150 V were supplied for a time span of 0.1-5 ms.

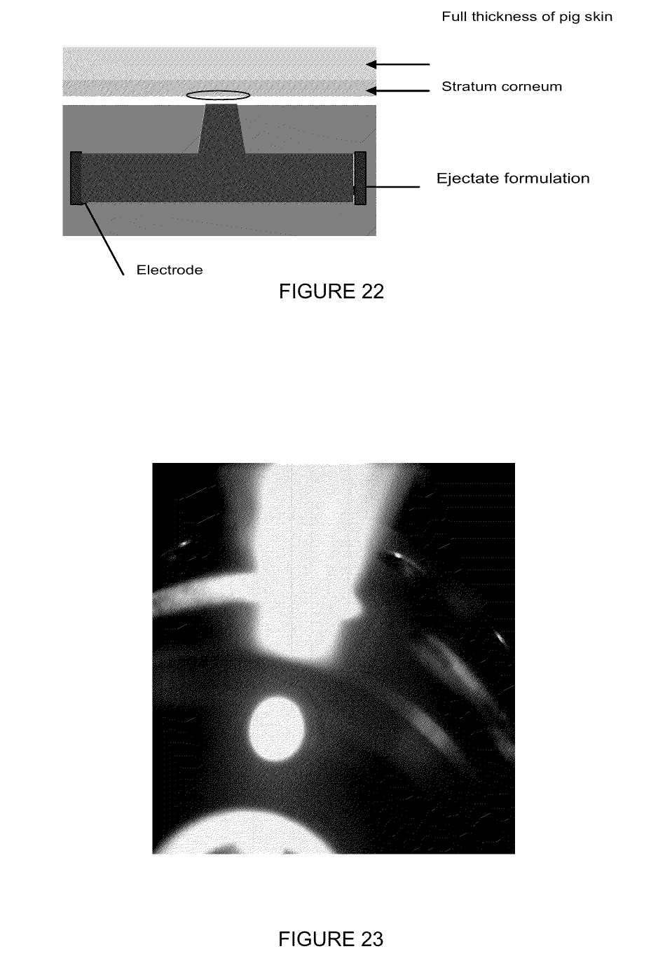

As shown in FIGS. 21 and 22, a microdevice was designed and fabricated to produce microsecond microjets. The design included a 1-8 mm³ microchamber having two brass electrodes on each side and a micronozzle on the front face with a radius of 25-200 μm; and a capacitor discharge power supply connected to the electrodes. The microchamber was filled with deionized water or other formulations which serve as the ejectate material. The microdevice surface was covered with a mask made of PDMS with rectangular holes (100 μm×100 μm) aligned with the micronozzles to further localize the exposed area of skin.

As a first assessment of the performance of this microdevice, the microdevice was activated while imaging, using high-speed microscopic photography. FIG. 23 shows an image of the jet and flash of light emitted from the microdevice upon activation, which validated the expectation that this approach could expel a microjet of fluid. The flash of light was consistent with the expected arc-based mechanism.

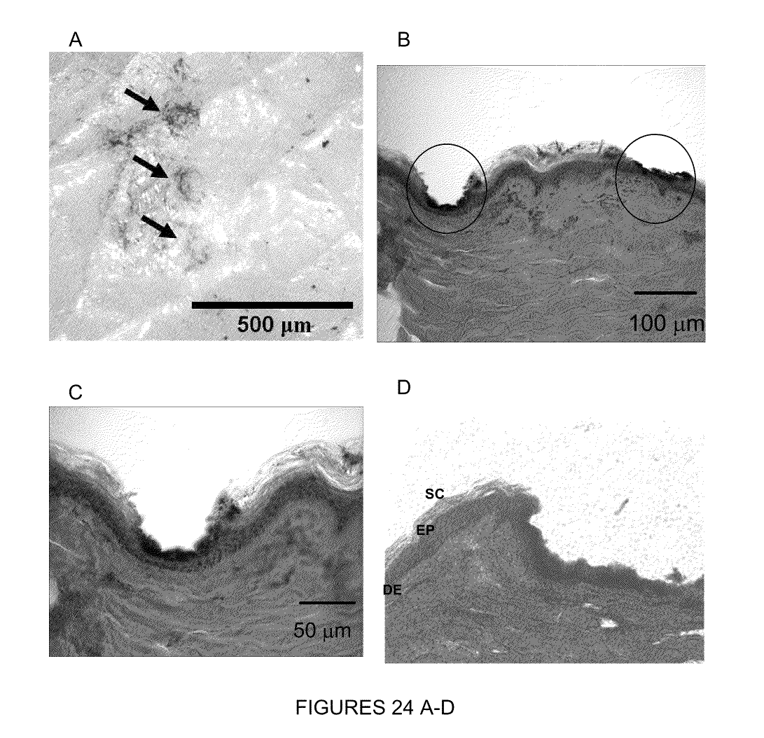

The arc-based ablation device was then activated in contact with human and porcine cadaver skin and both imaged the skin and measured its permeability to calcein. To perform histological analysis, full-thickness, shaved swine skin was placed onto the microdevice and exposed to a single ablation. Tissue samples were then cryo-sectioned, stained with hematoxylin and eosin, and imaged by brightfield microscopy. After ablating skin with the arc-based microdevice, histological examination showed highly selective removal of stratum corneum, as shown in FIG. 24. FIG. 24 A shows an en face image of skin ablated at three adjacent locations. FIG. 24 B shows a histological cross section of skin ablation at two adjacent sites from a different skin sample. FIG. 24 C shows a further magnified view of one of these ablation sites. FIG. 24 D shows a still greater magnification of the edge of an ablation site from another skin sample. These representative images show highly localized ablation that completely removed the stratum corneum (SC), which is critically important for increased skin permeability, but does not appear to damage the viable epidermis (EP) or dermis (DE).

In some cases, the tissue looks black at the site of skin ablation. This discoloration does not appear to be due to, for example, tissue combustion or charring. Instead, x-ray photoelectron spectroscopy analysis of the skin surface determined that the black spots are small deposits of brass debris from the microdevice electrodes, which were ejected after being melted by the arc discharge. This artifact can be reduced by using different kinds of metal electrodes that have higher melting points, such as nickel (1455° C.) or platinum (1750° C.), instead of brass (900° C.).

The micro-nozzle and PDMS mask used to control the size of the skin ablation sites effectively guided the removal of stratum corneum in a highly localized manner. Corresponding to the 100 μm×100 μm mask size, the holes generated in the skin measured approximately 100 μm in size. The size of these holes could be changed by simply changing the size of the masking holes on the microdevice.

In these experiments, ablation was carried out using an arcing voltage of 100-150 V. In Voltages less than 100 V in these experiments were insufficient to remove stratum corneum. Above this threshold voltage, a significant dependence of skin ablation on arcing voltage up to 200 V, the highest voltage that could be applied by the apparatus as designed for these experiments, was not observed.

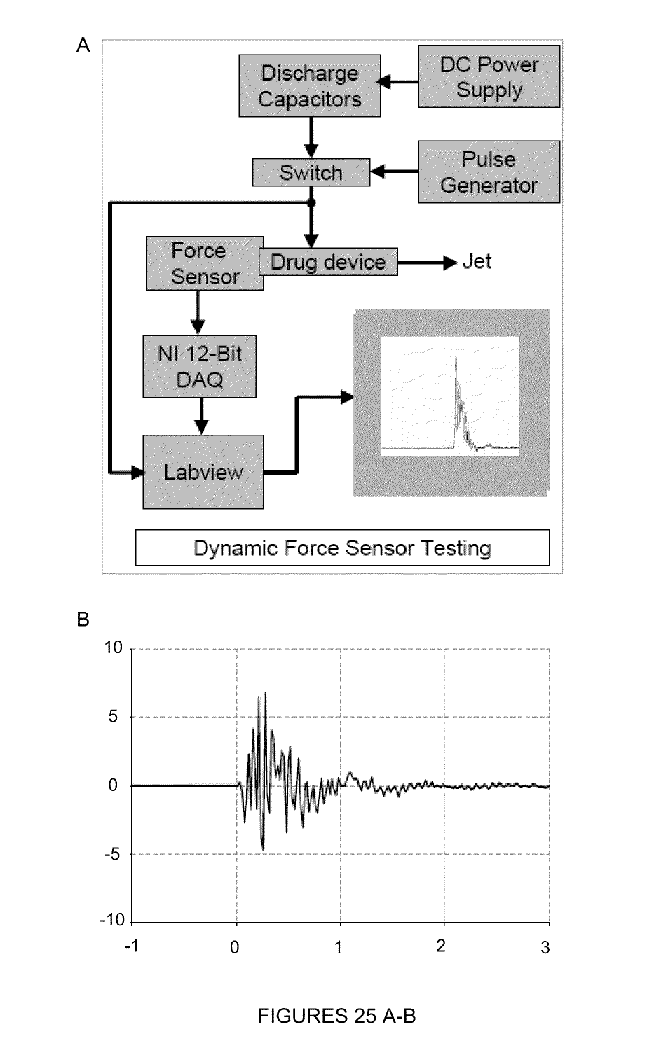

To determine the duration of the arcing and resulting microjet ejection, the electrical current in the capacitive discharge circuit is monitored across the MOSFET switch. Simultaneously, the recoil force of the microdevice during jet ejection is measured. The apparatus and method are explained in FIG. 25 A was utilized to measure the recoil force of the jet. A piezoelectric force sensor is incorporated into the apparatus to yield time-resolved force data during the discharge. The force sensor comprises two parts: a highly sensitive piezoelectric force sensor and a force amplifier. The sensor senses the extent of force generated from the released jet and the amplifier converts this force to a proportional electric charge which is then recorded using a data acquisition system. Both measurements indicated that the arcing and microjet ejection occurred on a timescale of 100 μs. The force generated from the ejected jet is measured to be approximately 1-10 N. (FIG. 25 B).

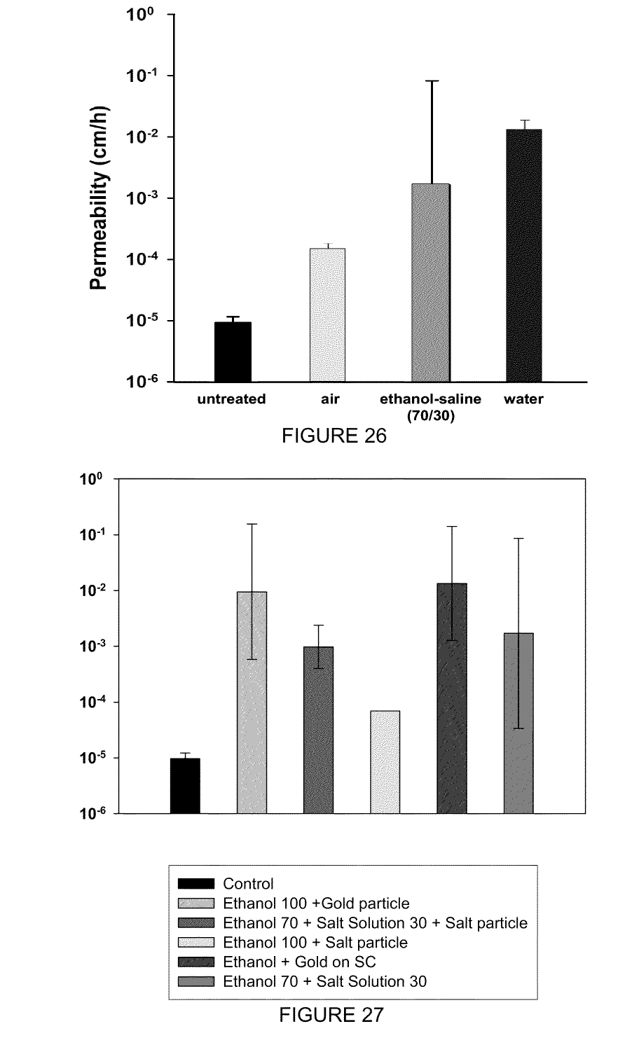

The observed efficient removal of stratum corneum should increase skin permeability. In order to measure skin permeability, heat-stripped, human epidermis was ablated using the microdevice and then placed in a Franz diffusion cell containing a model drug, calcein, in the donor compartment, which was assayed by spectrofluorometry. FIG. 26 shows permeability measurements made for delivery of calcein across human cadaver skin. For untreated skin, this permeability was just 10⁻⁵ cm/h, because calcein is a relatively large (623 Da), hydrophilic compound. After arc ablation of the skin with water, the permeability increased by 1000 fold to a value of 10⁻² cm/h. This large increase in skin permeability is highly significant for drug delivery applications.

Arc ablation with an ethanol-saline formulation similarly increased skin permeability, but to a lesser extent. Arc ablation with an empty (i.e., air-filled) micro-chamber also increased skin permeability, but only by a factor of 10. Arc ablation with water ejected from the microchamber was probably more effective because it more efficiently transferred heat (and momentum) to the tissue as compared to air.

Overall, this first study of skin ablation using an arc-discharge microdevice removed stratum corneum in a rapid and highly targeted manner. The depth of ablation was limited to the stratum corneum layer and the affected skin area was controlled on the micron scale by device design. In this manner, skin permeability to a hydrophilic model drug was increased by 1000 fold, which may enable transdermal delivery of a variety of compounds using this platform technology.

Additional formulations that could have different effects on skin permeability were also considered. Skin ablation depends on the energy generated by arcing. The electrical and chemical properties of the filling material could determine the power of arcing resulting in ablation. Several formulations of filling material were tested and skin permeability was compared. As shown in FIG. 27, the use of (i) ethanol with gold microparticles, (ii) ethanol-saline with salt microparticles, (iii) ethanol with gold microparticles placed on the skin surface, and (iv) ethanol-saline (without microparticles) all increased skin permeability by 100-1000 fold. It was hypothesized that such microparticles could be important because they (i) could increase the conductivity of the microjet formulation, and/or (ii) could act as projectiles jetted at the skin. Statistical testing, however, showed no statistical difference between these four formulations, given the large error bars. The final formulation, ethanol with salt particles, produced a significantly lower increase in permeability.

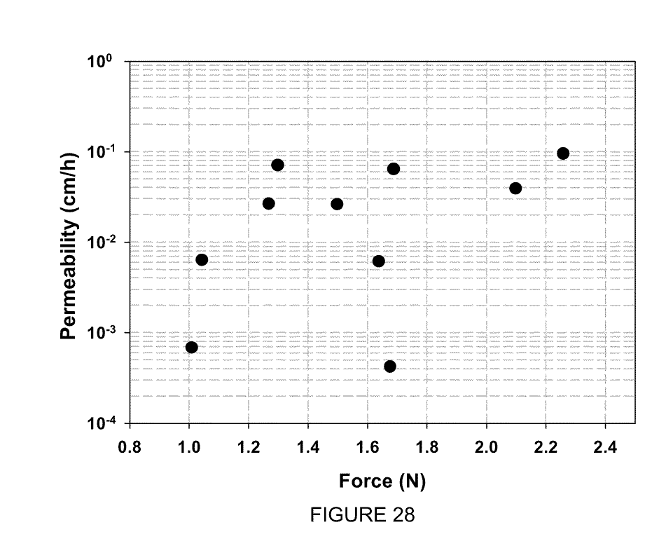

It was hypothesized that the variability in skin permeability could be explained because skin permeability might correlate with the force of microjet ejection from the microdevice. To test this hypothesis, the reaction force of the microdevice during ablation was measured and plotted versus the increase in skin permeability for a series of experiments carried out using a saline solution as the microreservoir filling solution (e.g., medium). As shown in FIG. 28, there was no apparent correlation. This suggests that other aspects of the experimental apparatus were poorly controlled in this study, which was carried out during our first experiments using the arc-ablation method, such as contact distance and angle between the ablation device and the skin.

As a final aspect of this study, the temperature of the microjets ejected from the arc-based device was determined. A Ni—Cr thermocouple was placed inside the microreservoir to measure the temperature directly; however, this did not work because the thermocouple was damaged by the arcing process, perhaps by the high temperature, perhaps by direct interaction with the arc, and/or perhaps by the high pressure and velocity of the ejection. Our next approach was to use the temperature-indicator, liquid-crystal paper discussed above. Because it was similarly damaged by placement directly at the microjet orifice, the paper was placed beneath a 50-μm thick polymer film to protect it. Using this measurement technique, the temperature below the polymer film at the site of the liquid-crystal paper was determined to lie between 60 and 100° C. However, given the very short duration of the thermal pulse, there should be a steep temperature gradient across the polymer film. Preliminary computer calculations were conducted to estimate the temperature at the surface of the polymer film directly exposed to the microjet. This temperature was estimated to be hundreds of degrees Celsius and even greater than 500° C.

### Example 5

### Microthermal Ablation for Transdermal Drug Delivery

A micro device shown in FIGS. 2 A-B was designed and fabricated to generate the ablation energy. The system has a 1 μl microchamber having two metal electrodes on each side and a nozzle at top; and a power supply circuit with discharging capacitors providing the electrical energy to the electrodes. The microchamber was filled with PBS water serving as a medium to cause the arc discharge phenomenon. The metal mask covered the outer surface of the nozzle and additional masks were used to localize the skin ablation effect, as illustrated in FIGS. 3A and 3B.

The ablation energy generated by the system was simulated with the software COMSOL MULTIPHYSICS® and the temperature was measured by using thermal indicating papers coated with heat sensitive polymer film, which turns transparent and shows the color of the background upon reaching a designated temperature. The thermal paper was placed on the metal mask exposed to hot medium ejected from the system and the temperature of the hot medium was computed on the basis of the temperature measurement. The recoil force generated by arc discharge was measured by a force sensor installed at the back side of the microchamber. The force sensor recorded the change of the generated recoil force over time after triggering the arc discharge phenomenon in the microchamber.

For histological analysis, shaved pig cadaver skin was placed on the metal mask in the same way as when measuring the temperature. Then, a viscous drug solution containing a model drug, sulforhodamine, was mounted on the ablated skin surface to visualize the effect of the skin ablation on the drug permeation. Tissue samples were imaged by brightfield and fluorescent microscopy, cryo-sectioned, and stained with hematoxylin and eosin to observe the removal of stratum corneum.

In this example, the ablation system was operated using the voltage of 100-150 V, which was experimentally determined as the threshold voltage range to obtain the reliable skin ablation. The total energy stored in the system while supplying electrical energy was simulated between 4 and 5 J and the temperature of the water medium filled in the microchamber was computed up to 600° C. with the assumption that the energy generated by the arc discharge phenomenon between the two electrodes was all used to heat the total amount of medium in the microchamber. Compared to other ablation techniques using the energy range of 30-50 mJ, the amount of energy released from this system is higher almost by 2 orders of magnitude.

Because direct measurement of the temperature of the ejected medium was not easy due to the high speed of arcing within the microsecond timescale, the temperature of heat energy transferred through the metal mask was measured from the ejected hot medium. Table 1 demonstrates the phase change of the heat sensitive polymer film, where black and white circles designate the change and non-change at each temperature, respectively. The thickness of the tungsten (W), nickel (Ni), and titanium (Ti) masks is 25, 75, and 75 μm, respectively. As shown in Table 1, the temperature of heat energy through 25 μm W mask was measured up to 290° C., but 25 μm Ni and Ti masks were physically damaged showing a hole in them. This means that it was probable to interpret the color change of heat sensitive paper not only with the thermal effect but also with the mechanical impact. The thicker nickel and titanium mask (75 μm) did not show the mechanical damage but the temperature of heat energy through them was lower. Although the tested tungsten mask was thinner, it did not show a hole in itself, probably resulting from the melting or the mechanical impact. Based on the result of Table 1, the temperature of heat energy released from the system was simulated to be at least 1000° C.

| | Temp. (° C.) | | | | | |
|---|---|---|---|---|---|---|
| Metal (thickness) | 160 | 204 | 224 | 241 | 260 | 290 |
| W (25 μm) | • | • | • | • | • | • |
| Ni (75 μm) | • | • | • | • | • | ∘ |
| Ti (75 μm) | • | • | • | ∘ | ∘ | ∘ |

The recoil force generated by the system ranged from 1 to 2.5 N. With the force data, it is likely that the direct skin contact of the system can provide a synergistic effect of thermal and mechanical ablation by the medium ejected from the microchamber. The medium ejected from the microchamber created the recoil force and the record of recoil force was all completed within 100 μs. This timescale of measuring the recoil force is believed to be a similar time range of the heat ablation resulting from the heat energy transfer from the ejected medium to the skin while arcing occurs. With these results, the amount of heat energy transferred through the metal mask can provide enough energy to ablate stratum corneum on a micro-second (i.e., sub-millisecond) timescale.

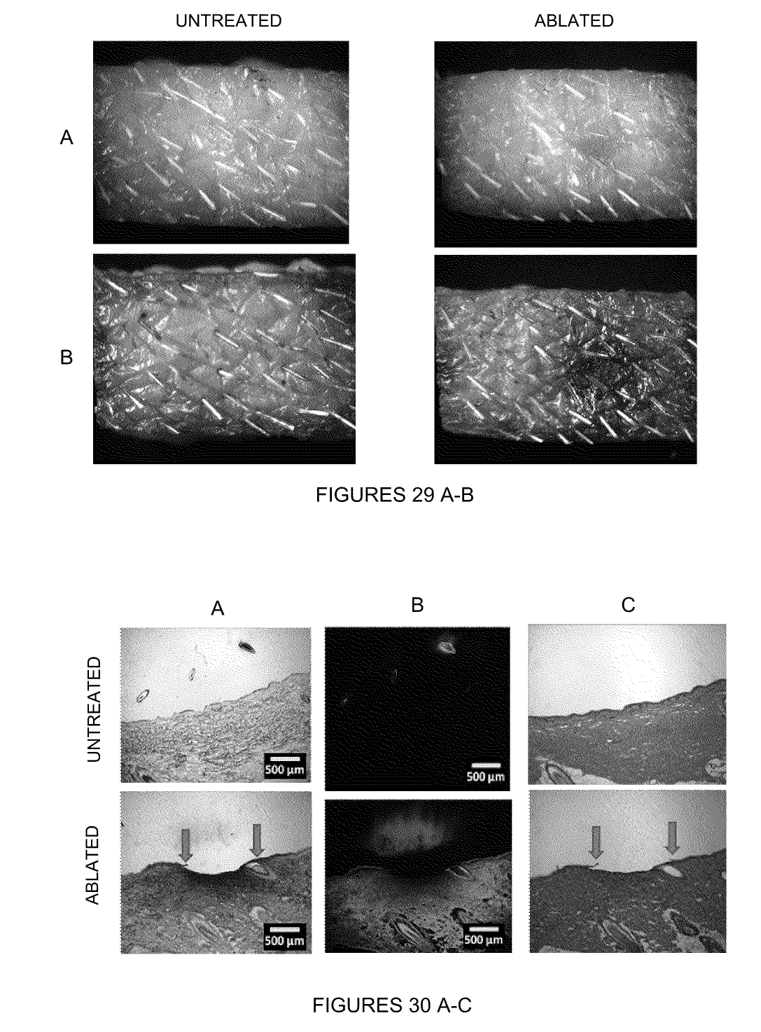

To illustrate that removal of the stratum corneum increases skin permeability, the delivery of a model drug, sulforhodamine, was examined. FIG. 29 A shows the surface of the untreated skin (control) and the ablated skin after applying the ablation system to the skin surface. FIG. 29 B shows the same sample after 12 h delivery of the model drug, sulforhodamine. Compared to the control sample, the skin surface around the ablated site showed the radially decreasing intensity of purple staining by sulforhodamine, indicating the diffusion of sulforhodamine into the skin.

This diffusion was identified with histological examination shown in FIG. 30. While the control sample shows no diffusion of hydrophilic sulforhodamine, the brightfield and fluorescent images (FIGS. 30A and B) of the ablated skin sample show a gradient in sulforhodamine and fluorescence from the ablated top to the deeper tissue due to the diffused sulforhodamine, respectively. FIG. 30 C shows a cross section of the stained skin sample, which previously had intact stratum corneum and which then lost stratum corneum without apparent viable epidermis damage. Furthermore, an additional mask having localizing windows enabled the separated and controlled effect of the heat ablation. Thus, the localization of the skin ablation may determine the release rate of drug by controlling the area where the diffusion occurs.

What is claimed is:

1. An ablation system for a surface, comprising:
an electric current generating system;
a propulsion system in operative communication with the electric current generating system; and
a medium, wherein the medium is propelled towards a surface by the propulsion system in response to an electric current generated by the electric current generating system, wherein the electric current does not contact the surface.

2. The ablation system of claim 1, wherein the electric current generating system comprises at least one chamber containing the medium, the at least one chamber comprising at least two electrodes configured to generate the electrical current therebetween.

3. The ablation system of claim 2, wherein the at least two electrodes of the at least one chamber are configured to permit an arc discharge therebetween.

4. The ablation system of claim 1, wherein the propulsion system comprises the at least one chamber having a nozzle, wherein the medium is propelled from the at least one chamber through the nozzle upon generation of a current.

5. The ablation system of claim 1, wherein the ablation system is capable of ablating the surface in less than about 100 microseconds.

6. The ablation system of claim 1, further comprising an interface layer, wherein the interface layer is provided between the ablation system and the surface.

7. The ablation system of claim 6, wherein the interface layer comprises regions having different heat transfer properties.

8. The ablation system of claim 1, wherein the surface is a biological surface.

9. The ablation system of claim 1, further comprising at least one active agent, wherein the at least one active agent is delivered to the surface.

10. The ablation system of claim 1, wherein the volume of each of the at least one chambers is less than about one milliliter.

11. The ablation system of claim 1, wherein the area of the surface ablated by each of the at least one chambers comprises less than about one millimeter.

12. A method for ablating a surface, comprising
providing an ablation apparatus to a surface;
generating an electric current with the ablation apparatus, wherein the electric current does not contact the surface;
ejecting a medium from the ablation apparatus towards the surface; and
ablating the surface.

13. The method for ablating a surface of claim 12, wherein generating a current comprises inducing an arc discharge.

14. The method for ablating a surface of claim 12, wherein the surface is a biological surface.

15. The method for ablating a surface of claim 14, wherein the biological surface is skin or a mucosal tissue.

16. The method for ablating a surface of claim 15, wherein ablating the surface comprises altering the stratum corneum.

17. The method for ablating a surface of claim 12, further comprising providing an interface layer between the ablation system and the surface.

18. The method for ablating a surface of claim 17, further comprising differentially transferring heat to different regions of the surface.

19. The method for ablating a surface of claim 12, further comprising delivering an active agent to a surface.

20. The method for ablating a surface of claim 12, wherein ablating the surface occurs in less than about 100 microseconds.
